(19) United States
(12) Patent Application Publication
(10) Pub. No.: US 2024/0400704 A1
(43) Pub. Date: Dec. 5, 2024

(54) OPTIMIZED MULTABODY CONSTRUCTS, COMPOSITIONS, AND METHODS

(71) Applicants: The Hospital for Sick Children, Toronto (CA); Radiant Biotherapeutics Inc., Montreal (CA)

(72) Inventors: Jean-Philippe Julien, Toronto (CA); Edurne Rujas Diez, Vitoria (ES); Joanne Hulme, Montreal (CA); Peter Edward Bayliss, Montreal (CA); Xinwen He, Montreal (CA); Melissa Beilschmidt, Montreal (CA)

(21) Appl. No.: 18/691,267

(22) PCT Filed: Sep. 13, 2022
(86) PCT No.: PCT/CA2022/051360

§ 371 Date: Mar. 12, 2024

Related U.S. Application Data
(60) Provisional application No. 63/243,402, filed on Sep. 13, 2021.

(51) Int. Cl. C07K 16/28; A61K 39/00; A61P 35/00; C07K 14/47

(52) U.S. Cl. CPC C07K 16/2878; A61P 35/00; C07K 14/47; A61K 2039/505; C07K 2317/55; C07K 2317/73; C07K 2317/92; C07K 2317/94; C07K 2319/30

(57) ABSTRACT

Optimized self-assembled poly peptide complexes comprising 1) fusion polypeptides comprising Fc polypeptides linked to nanocage monomers or subunits thereof and 2) fusion polypeptides comprising an antigen-binding antibody fragment.

### CROSS-REFERENCE TO RELATED APPLICATIONS

The present application claims the benefit of and priority to U.S. Provisional Application No. 63/243,402 filed Sep. 13, 2021, the entire content of which is hereby incorporated by reference in its entirety for all purposes.

### SEQUENCE LISTING

The instant application contains a Sequence Listing which has been submitted electronically in XML format and is hereby incorporated by reference in its entirety. Said XML file, created on Sep. 13, 2022, is named Sequence Listing September-2022 3206-5070_R and is 24.8 kilobytes in size.

### BACKGROUND

Therapeutics based on antibodies or antibody fragments are being developed for a variety of uses, e.g., for treating various diseases or conditions.

However, antibody-based therapeutics may need to be tuned so that they have desirable characteristics (for example, desirable biodistribution, half-lives, etc.) after administration to a subject. Some antibody-based therapeutics have a format that differs from that of a native immunoglobulin molecule. For example, in some therapeutics, an antibody or antibody fragment is fused to another polypeptide: in some, the antibody or antibody fragment(s) is in a configuration or has a valence not found in nature. These antibody-based therapeutics may also need to be tuned.

Thus, there remains a need for optimized antibody-based therapeutics.

### SUMMARY

The present invention addresses this need with the provision of self-assembled polypeptide complexes comprising (a) fusion polypeptides (1) comprising Fc polypeptides and (2) a nanocage monomer or subunit thereof and (b) fusion polypeptides (1) comprising an antibody fragment and (2) a nanocage monomer or subunit thereof. In certain embodiments, the Fc polypeptides comprise certain amino acid residues at particular positions.

In one aspect, provided are fusion polypeptides comprising:
(1) an Fc polypeptide and
(2) a nanocage monomer or subunit thereof, wherein the Fc polypeptide comprises an IgG1 Fc chain, wherein said IgG1 Fc chain comprises (1) an amino acid residue other than glycine at position 237 and (2) a proline residue at position 329, according to EU numbering.
In some embodiments, the nanocage monomer or subunit thereof is a ferritin monomer or subunit thereof.

In some embodiments, the ferritin monomer or subunit thereof is a ferritin light chain or subunit thereof.

In some embodiments, the ferritin monomer or subunit thereof is a human ferritin or subunit thereof.

In some embodiments, the ferritin monomer or subunit thereof is a ferritin monomer subunit, e.g., a C-half ferritin. In some such embodiments, the Fc polypeptide is linked to the C-half ferritin's N-terminus, e.g., via an amino acid linker. In some embodiments, the amino acid linker comprises a (GₙS)ₘ linker, e.g., a (GGGGS)ₘ(SEQ ID NO:19) linker.

In some embodiments, the Fc polypeptide comprises a single chain Fc (scFc) comprising two Fc chains, wherein the two Fc chains are linked via an amino acid linker, e.g., a (GₙS)ₘ linker, e.g., a (GGGGS)ₘ(SEQ ID NO:19) linker.

In some embodiments, the Fc polypeptide comprises an IgG1 Fc chain.

In some embodiments, the IgG1 Fc chain comprises an alanine at position 237 according to EU numbering.

In some embodiments, the IgG1 Fc chain comprises: an alanine at position 234, an alanine at position 235, an arginine at position 236, and a leucine at position 330, according to EU numbering.

In one aspect, provided are self-assembled polypeptide complexes comprising:
(a) a plurality of first fusion polypeptides, each first fusion polypeptide being a fusion polypeptide of any one of claims 1-16, and
(b) a plurality of second fusion polypeptides, each second fusion polypeptide comprising (1) an antigen-binding antibody fragment and a (2) a nanocage monomer or subunit thereof.

In some embodiments, the nanocage monomer or subunit thereof of each second fusion polypeptide is a ferritin monomer or subunit thereof.

In some embodiments, the ferritin monomer or subunit thereof is a ferritin light chain or subunit thereof.

In some embodiments, the ferritin monomer or subunit thereof is a human ferritin or subunit thereof.

In some embodiments, the self-assembled polypeptide complex does not comprise any ferritin heavy chains or subunits of ferritin heavy chains.

In some embodiments, within each second fusion polypeptide, the antigen-binding antibody fragment is linked to the nanocage monomer or subunit thereof via an amino acid linker, e.g., an amino acid linker comprising a (GₙS)ₘ linker, e.g., a (GGGGS)ₘ(SEQ ID NO: 19) linker.

In some embodiments, the antigen-binding antibody fragment of each second fusion polypeptide is linked to the N-terminus of nanocage monomer or subunit thereof.

In some embodiments, the antigen-binding antibody fragment of each second fusion polypeptide is a Fab fragment.

In some embodiments, each second fusion polypeptide does not comprise any antibody CH2 or CH3 domains.

In some embodiments, the self-assembled polypeptide complex, further comprises a plurality of third fusion polypeptides, each third fusion polypeptide comprising (1) an antigen-binding antibody fragment and (2) a nanocage monomer or a subunit thereof, wherein the third fusion polypeptide is different than the second fusion polypeptide. In some embodiments, the antigen-binding antibody fragment of each third fusion polypeptide is a Fab fragment. In some embodiments, each third fusion polypeptide does not comprise any antibody CH2 or CH3 domains.

In some embodiments, the nanocage monomer or subunit thereof of each first fusion polypeptide and each second fusion polypeptide is a ferritin monomer subunit, and
a. each first fusion polypeptide comprises a C-half-ferritin, and each second fusion polypeptide comprises a N-half-ferritin; or
b. each first fusion polypeptide comprises an N-half ferritin, and each second fusion polypeptide comprises a C-half-ferritin.

In some embodiments, the self-assembled polypeptide complex is characterized by a 1:1 ratio of first fusion polypeptides to second fusion polypeptides.

In some embodiments, the self-assembled polypeptide complex comprises a total of 24 to 48 fusion polypeptides. In some embodiments, the self-assembled polypeptide complex comprises a total of least 24 fusion polypeptides.

In some embodiments, the self-assembled polypeptide complex comprises a total of at least 32 fusion polypeptides.

In some embodiments, the self-assembled polypeptide complex has a total of about 32 fusion polypeptides.

In some embodiments, the self-assembled polypeptide complex exhibits no binding to at least one human Fcγ receptor, as determined in an in vitro assay.

In some embodiments, the self-assembled polypeptide complex exhibits no binding to one or more human Fcγ receptors selected from the group consisting of hFcγRI, hFcγRIIa, hFcγRIIb, hFcγRIIIa, hFcγRIIIb, and combinations thereof, as determined in an in vitro assay. In some embodiments, the self-assembled polypeptide complex exhibits no binding to hFcγRI, as determined in an in vitro assay. In some embodiments, the self-assembled polypeptide complex exhibits no binding to hFcγRIIa, as determined in an in vitro assay. In some embodiments, the self-assembled polypeptide complex exhibits no binding to hFcγRIIIa, as determined in an in vitro assay. In some embodiments, the self-assembled polypeptide complex exhibits no binding to hFcγRIIb, as determined in an in vitro assay. In some embodiments, the self-assembled polypeptide complex exhibits no binding to hFcγRIIIb, as determined in an in vitro assay.

In one aspect, provided are self-assembled polypeptide complexes comprising:
(a) a plurality of first fusion polypeptides, each first fusion polypeptide comprising (1) a scFc and (2) a ferritin monomer or subunit thereof, and
(b) a plurality of second fusion polypeptides, each second fusion polypeptide comprising (1) an antigen-binding antibody fragment linked to (2) a ferritin monomer or subunit thereof, wherein the scFc comprises two IgG1 Fc chains, each IgG1 Fc chain comprising: an alanine at position 234, an alanine at position 235, an arginine at position 236, an alanine at position 237, a proline at position 329, and a leucine at position 330, according to EU numbering.

In some embodiments,
a. each first fusion polypeptide comprises a C-half-ferritin, and each second fusion polypeptide comprises an N-half-ferritin: or
b. each first fusion polypeptide comprises an N-half ferritin, and each second fusion polypeptide comprises a C-half-ferritin.

In some embodiments, each first fusion polypeptide comprises a scFc linked to the C-half-ferritin's N-terminus, and each second fusion polypeptide comprises a Fab linked to the N-half-ferritin's N-terminus.

In some embodiments, (1) in each first fusion polypeptide, the scFc is linked via an amino acid linker to the C-half ferritin's N-terminus and/or (2) in each second fusion polypeptide, the Fab is linked via an amino acid linker to N-half ferritin's N-terminus.

In some embodiments, the self-assembled polypeptide complex is characterized by a 1:1 ratio of first fusion polypeptides to second fusion polypeptides.

In some embodiments, the self-assembled polypeptide complex further comprises a plurality of third fusion polypeptides, each third fusion polypeptide comprising (1) an antigen-binding antibody fragment linked to (2) a nanocage monomer or a subunit
thereof, wherein the third fusion polypeptide is different than the second fusion polypeptide.

In some embodiments, the self-assembled polypeptide complex comprises a total of 24 to 48 fusion polypeptides.

In some embodiments, the self-assembled polypeptide complex comprises a total of least 24 fusion polypeptides. In some embodiments, the self-assembled polypeptide complex comprises a total of at least 32 fusion polypeptides. In some embodiments, the self-assembled polypeptide complex has a total of about 32 fusion polypeptides.

In some embodiments, the antibody-binding fragment is capable of binding to a tumor-associated antigen or an antigen associated with an autoimmune disorder.

In one aspect, provided are methods comprising administering a composition comprising the self-assembled polypeptide complex of any one of claims 17-54 to a mammalian subject. In some embodiments, the subject is human. In some embodiments, the subject has or is at risk of developing cancer. In some embodiments, the subject has or is at risk of developing an autoimmune disorder.

In some embodiments, the method comprises administration by a systemic route. In some embodiments, the systemic route comprises subcutaneous, intravenous, or intramuscular injection, inhalation, or intranasal administration.

In one aspect, provided are uses of a composition comprising the self-assembled polypeptide complex described herein for administration to a mammalian subject.

In some embodiments, the subject is human.

In some embodiments, the subject has or is at risk of developing cancer.

In some embodiments, the subject has or is at risk of developing an autoimmune disorder.

In some embodiments, the composition is for administration by a systemic route.

In some embodiments, the systemic route comprises subcutaneous, intravenous, or intramuscular injection, inhalation, or intranasal administration.

In one aspect, provided are compositions comprising the self-assembled polypeptide complex described herein for use in administration to a mammalian subject.

In some embodiments, the subject is human.

In some embodiments, the subject has or is at risk of developing cancer.

In some embodiments, the subject has or is at risk of developing an autoimmune disorder.

In some embodiments, the compositions are for administration by a systemic route.

In some embodiments, the systemic route comprises subcutaneous, intravenous, or intramuscular injection, inhalation, or intranasal administration.

### BRIEF DESCRIPTION OF THE DRAWINGS

FIG. 1A is a diagrammatic representation of human ferritin light chain (hFTL) and exemplary N-half ferritin (N-hFTL) and C-half ferritin (C-hFTL) molecules.

FIG. 1B is a diagrammatic representation of fusion polypeptides that together form exemplary Multabodies of the disclosure.

FIGS. 2A, 2B, 2C, 2D, and 2E depict biolayer interferometry (BLI) time-response curves for the binding of a DR5-targeting Multabody (Cona MB IgG1 LLRAL) to human DR5, human DR4, human osteoprotegerin (OPG), human decoy receptor 1 (DcR1), and human DcR2, respectively.

FIG. 3 depicts exemplary BLI time-response curves for the binding of DR5-targeting Multabodies containing various Fc chains to human, cynomolgus monkey, and mouse FcRns, measured at pH 6.0 for association and pH 7.4 for dissociation.

FIG. 4 illustrates the dose-dependent killing of cancer cells by Cona MB IgG1 wt, Cona MB IgG1 LLRAL, and conatumumab (Cona) in different human tumor cell lines, quantified and represented as the percentage viable cells following Multabody or antibody treatment to vehicle-treated tumor cells.

FIG. 5A is a schematic illustrating the designs of pharmacokinetic studies described in Example 6.

FIG. 5B depict plots showing the plasma levels of Cona MB IgG1 wt, Cona MB IgG1 LLRAL, or conatumumab (Cona) following a single intraperitoneal (i.p.) or intravenous (i.v.) dose administered to severe combined immunodeficiency (SCID) mice.

FIG. 5C illustrates plasma levels of Cona MB IgG1 LLRAL or conatumumab in SCID mice administered with two i.p. doses, 96 hours apart.

FIG. 6A is a schematic that illustrates the design of an in vivo efficacy experiment described in Example 7 and conducted in a COLO 205 xenograft mouse model.

FIGS. 6B and 6C show tumor volumes at various timepoints (FIG. 6B) and on Day 88 (FIG. 6C) in mice bearing COLO 205 xenograft tumors and treated with vehicle, conatumumab, or Cona MB IgG1 LLRAL.

FIGS. 6D-6G shows tumor growth curves for individual COLO 205 xenograft tumor-bearing mice treated with vehicle (FIG. 6D), conatumumab (FIG. 6E), or Cona MB IgG1 LLRAL (FIGS. 6F and 6G).

FIG. 6H is a time plot that depicts plasma levels of Cona MB IgG1 LLRAL or conatumumab following i.p. administration to COLO 205 xenograft tumor-bearing mice.

FIG. 7A is a plot that depicts tumor volumes of mice in various groups over time following the first dose. “MB” indicates the Cona MB IgG1 LLRAL group. The triangles on the x-axis denote the treatment time points. FIGS. 7B-7F show the tumor volumes of individual mice within the vehicle (FIG. 7B) and 5 mg/kg, 1 mg/kg. 0.25 mg/kg, and 0.1 mg/kg Cona MB IgG1 LLRAL treatment (FIG. 7C, FIG. 7D, FIG. 7E, and FIG. 7F, respectively) groups. In FIGS. 7B-7F, “CR” denotes complete regression.

FIG. 7G is a plot that depicts that amount of Cona MB IgG1 LLRAL detectable in blood samples collected at various timepoints after the first dose was administered. Cona MB IgG1 LLRAL was detectable at all time points tested in the 0.1 mg/kg. 0.25 mg/kg, 1 mg/kg/and 5 mg/kg Cona MB IgG1 LLRAL treatment groups, with the pharmacokinetics appearing linear in all dose groups.

FIG. 8 is a schematic depicting the design of a study of large tumor penetration and apoptosis induction by a DR5-targeting Multabody in an COLO 205 xenograft mouse model. Experiments are described in Example 9.

FIGS. 9A-9D depict representative tumor sections from mice that were untreated (FIGS. 9A and 9C) or treated with a DR5-targeting Multabody (“MB”) (FIGS. 9B and 9D). Sections were stained with an antibody against cleaved caspase-3, a marker of apoptosis. Experiments are described in Example 9.

### DETAILED DESCRIPTION OF CERTAIN EMBODIMENTS OF THE INVENTION

### Definitions

The terms “about” and “approximately,” when used herein in reference to a value, are used interchangeably and refer to a value that is similar to the referenced value. In general, those skilled in the art, familiar with the context, will appreciate the relevant degree of variation encompassed by “about” or “approximately” in that context. For example, in some embodiments, the terms “about” and “approximately” may encompass a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the terms “alter,” “altered,” “decrease,” “decreased,” “increase,” “increased,” or “reduction,” “reduced,” (e.g., in reference to certain outcomes or effects) have meanings relative to a reference level. In some embodiments, in the context of discussing mutations in an Fc chain or Fc polypeptide, the reference level is a level known or as determined with an IgG that does not contain the referenced mutation(s) in the Fc region.

As used herein, the term “binding.” unless otherwise specified, refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). As used herein, the phrases “non-binding” or “no binding,” or similar phrases, between two entities refers to 1) a lack of detectable binding or 2) binding below a set threshold that corresponds to no binding in an appropriate assay, e.g., an in vitro binding assay such as biolayer interferometry. For example, in some embodiments, in an in vitro biolayer interferometry assay, a maximal association binding response of less than 0.1 nm after 180 seconds to a biosensor loaded with 0.8 nm of target when the test article is present at a concentration of 20 nM is classified as “non-binding.”

The terms “ferritin” and “apoferritin” are used interchangeably herein and generally refer to a polypeptide (e.g., a ferritin chain) that is capable of assembling into a ferritin complex which typically comprises 24 protein subunits. In some embodiments, the ferritin is a human ferritin, e.g., a human ferritin light chain, e.g., a human ferritin light chain having at least 85% sequence identity to SEQ ID NO: 1 or UniProt P02792. In some embodiments, the ferritin is a wild-type ferritin. For example, the ferritin may be a wild-type human ferritin.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains.

As used herein, the term “linker” is used to refer to an entity that connects two or more elements to form a multi-element agent. For example, those of ordinary skill in the art appreciate that a polypeptide (e.g., fusion polypeptide) whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, the linker is an “amino acid linker,” that is, it comprises amino acid residues, e.g., an amino acid linker may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acid residues. In some embodiments, a linker is characterized in that it tends not to adopt a rigid three-dimensional structure, but rather provides flexibility to the polypeptide.

The term “multispecific,” as used herein, refers to the characteristic of having at least two binding sites at which at least two different binding partners, e.g., an antigen or receptor (e.g., Fc receptor), can bind. For example, a polypeptide complex that comprises at least two Fab fragments, wherein each of the two Fab fragments is capable of binding to a different antigen, is “multispecific.” As an additional example, a polypeptide complex that comprises an Fc fragment (which is capable of binding to an Fc receptor) and a Fab fragment (which is capable of binding to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to at least two binding sites may be the same or different.

The term “nanocage monomer,” as used herein, refers to a single chain of a polypeptide that is capable of self-assembling with other nanocage monomers to form a self-assembled polypeptide complex comprising a plurality of nanocage monomers. In some embodiments, the nanocage monomer is selected from monomers of ferritin, apoferritin, encapsulin, sulfur oxygenase reductase (SOR), lumazine synthase, pyruvate dehydrogenase, carboxysome, vault proteins, GroEL, heat shock protein, E2P coat protein, MS2 coat protein, fragments thereof, and variants thereof.

The term “polypeptide,” as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, glycosylation etc. In some embodiments, proteins may comprise natural amino acids, non-natural amino acids, synthetic amino acids, and combinations thereof.

The term “self-assembled,” when used in reference to a macromolecular complex (e.g., a polypeptide complex), refers to the spontaneous formation of that complex when sufficient constituents of the complex (e.g., fusion polypeptides) to be formed are present. In some embodiments, complexes self-assemble in physiological conditions, or in a buffer (e.g., a solution) that corresponds to physiological conditions.

As used herein, the term “subject” to an organism, typically a mammal (e.g., a human). In some embodiments, a subject is suffering from or susceptible to a relevant disease, disorder or condition. In some embodiments, a subject displays one or more symptoms or characteristics of a disease, disorder or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the term “tumor-associated antigen or “tumor associated antigen” means an antigen that is present on tumor cells at a significantly greater amount than on normal cells.

### A. Fusion Polypeptides

In many embodiments, fusion polypeptides compatible with compositions and methods disclosed herein generally comprise a nanocage monomer or subunit thereof linked to either an Fc polypeptide or to an antigen-binding antibody fragment. Within the fusion polypeptide, the Fc polypeptide or the antigen-binding antibody fragment may be linked to the nanocage monomer or subunit thereof at a particular terminus of the nanocage monomer or subunit thereof, e.g., the N-terminus or the C-terminus. In some embodiments, the Fc polypeptide or antigen-binding antibody fragment is linked via an amino acid linker, such as a linker as described herein.

### 1. Nanocage Monomers and Subunits Thereof

In some embodiments, the nanocage monomer is a ferritin monomer.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains. In some embodiments, the ferritin monomer is a ferritin light chain. In some embodiments, the ferritin monomer does not include a ferritin heavy chain or other ferritin components capable of binding to iron or capable of ferroxidase activity.

In some embodiments, each fusion polypeptide within the self-assembled polypeptide complex comprises a ferritin light chain or a subunit of a ferritin light chain. In these embodiments, the self-assembled polypeptide complex does not comprise any ferritin heavy chains or subunits of ferritin heavy chains.

In some embodiments, the ferritin monomer is a human ferritin chain, e.g., a human ferritin light chain, e.g., a human ferritin light chain having the sequence of at least residues 2-175 of SEQ ID NO: 1.

A “subunit” of a ferritin monomer refers to a portion of a ferritin monomer that is capable of spontaneously associating with another, distinct subunit of a ferritin monomer, so that the subunits together form a ferritin monomer, which ferritin monomer, in turn, is capable of self-assembling with other ferritin monomers to form a polypeptide complex.

In some embodiments, the ferritin monomer subunit comprises approximately half of a ferritin monomer. As used herein, the term “N-half ferritin” refers to approximately half of a ferritin chain, which half comprises the N-terminus of the ferritin chain. As used herein, the term “C-half ferritin” refers to approximately half a ferritin chain, which half comprises the C-terminus of the ferritin chain. The exact point at which a ferritin chain may be divided to form the N-half ferritin and the C-half ferritin may vary depending on the embodiment. In the context of ferritin monomer subunits based on human ferritin light chain, for example, the halves may be divided at a point that corresponds to a position between about position 75 to about position 100 of SEQ ID NO:1 (or a substantial portion thereof). For example, in some embodiments, an N-half ferritin based on a human ferritin light chain has an amino acid sequence corresponding to residues 1-95 of SEQ ID NO: 1 (or a substantial portion thereof, e.g., residues 2-95 of SEQ ID NO: 1), and a C-half ferritin based on a human ferritin light chain has an amino acid sequence corresponding to residues 96-175 of SEQ ID NO: 1 (or a substantial portion thereof).

In some embodiments, the halves are divided at a point that corresponds to a position between about position 85 to about position 92 of SEQ ID NO: 1. For example, in some embodiments, an N-half ferritin based on a human ferritin light chain has an amino acid sequence corresponding to residues 1-90 of SEQ ID NO: 1 (or a substantial portion thereof, e.g., residues 2-90 of SEQ ID NO: 1), and a C-half ferritin based on a human ferritin light chain has an amino acid sequence corresponding to residues 91-175 of SEQ ID NO: 1 (or a substantial portion thereof.

### 2. Fc Polypeptides

In certain embodiments, fragment crystallizable (Fc) polypeptides comprise Fc chains that each have one or more mutations relative to a reference Fc chain of the same Ig class. As explained further herein below, the reference Fc chain may be of, e.g., the IgG1 class.

Unless otherwise noted, numbering of residues within an antibody fragment, e.g., an Fc chain, throughout this disclosure is according to the EU numbering.

In some embodiments, the Fc polypeptide comprises one or more human IgG1 Fc chains that is, except for mutations noted herein, the Fc polypeptide comprises an Fc chain that is substantially similar to that of the Fc chains within a wild type human IgG1.

In some embodiments, the Fc polypeptide comprises one or more IgG1 Fc chains (e.g., human IgG1 Fc chains or a human Fc chains), that is, except for having particular residue(s) (which may be different than the residue(s) in the corresponding wild type Fc chains) at certain positions as noted herein, the Fc polypeptide comprises an Fc chain that has an amino acid sequence that is substantially similar to that of the chains within a wild type IgG1 Fc. In some embodiments, the wild type IgG1 Fc is a human IgG1 Fc, in which each Fc chain has an amino acid sequence of SEQ ID NO:4.

For example, an Fc polypeptide may comprise an Fc chain with an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc chain within a wild-type IgG1 Fc. In some embodiments, an Fc polypeptide comprises an Fc chain that comprises the particular residue(s) at certain position(s) specifically described for that Fc chain, but has an amino acid sequence that is otherwise 100% identical to a corresponding Fc chain within a wild type Fc chain, e.g., wild type IgG1 Fc chain. In some embodiments, the Fc polypeptide comprises an Fc chain that has an amino acid sequence that differs by at least one, at least two, at least three, or at least four amino acid residues from the sequence of SEQ ID NO:4. In some embodiments, the Fc polypeptide comprises an Fc chain that has an amino acid sequence that differs by no more than ten, no more than nine, no more than eight, no more than seven, no more than six, no more than five, or no more than four amino acid residues from the sequence of SEQ ID NO:4.

In some embodiments, the Fc polypeptide is a single chain Fc (scFc), which comprises two Fc chains linked together by a covalent linker, e.g., via an amino acid linker.

In certain embodiments, the Fc chain comprises (1) an amino acid residue other than glycine at position 237; and (2) a proline residue at position 329. In some embodiments, the Fc chain comprises an alanine at position 237.

In some embodiments, the Fc chain is an IgG1 Fc chain and further comprises a mutation or set of mutations at one or more positions selected from position 234, position 235, position 236, position 330, and combinations thereof.

For example, in some embodiments, the Fc chain is an IgG1 Fc chain that comprises an alanine at position 234, an alanine at position 235, an arginine at position 236, an alanine at position 237, a proline at position 329, and a leucine at position 330.

In some embodiments, the Fc chain further comprises a mutation at a position associated with glycosylation, e.g., position 297 (e.g., by comprising a glutamine at position 297).

In some embodiments, the Fc chain comprises a mutation or set of mutations (relative to a corresponding wild type Fc chain) associated with an altered characteristic as further described herein. By “associated with,” it is meant that the mutation or set of mutations has been previously characterized, in the context of antibodies such as IgG antibodies, as conferring the altered characteristic (e.g., altered binding to FcRn, altered effector function, etc.) By “altered” it is meant that the characteristic (e.g., binding to an Fc receptor (e.g., an Fcγ receptor or an FcRn)), is different than that observed without the mutation or set of mutations.

For example, in some embodiments, the altered characteristic comprises altered binding to an Fc receptor.

In some embodiments, the altered characteristic comprising altered binding to an Fcγ receptor, e.g., a human FcγR. In some embodiments, the FcγR is a human FcγR selected from the group consisting of hFcγRI, hFcγRIIa, hFcγRIIb, hFcγRIlla, hFcγRIIIb, and combinations thereof.

In some embodiments, the altered binding comprises no binding, or significantly reduced binding, relative to a corresponding control (e.g., binding levels typically observed under similar circumstances with a corresponding wild type chain), in an assay, e.g., an in vitro assay.

### 3. Antibody Fragments

In certain embodiments, the antibody fragment is capable of binding to a tumor-associated antigen or an antigen associated with an autoimmune disorder.

In some embodiments, the antibody fragment is a Fab. In some embodiments, the antibody fragment is a single-chain Fab (scFab); for example, a fusion polypeptide comprising both the heavy and light chains of a Fab, optionally linked by a linker (e.g., amino acid linker as disclosed herein) is used.

In certain embodiments, the antibody fragment comprises a heavy chain variable region (e.g., a V_{H}). In certain embodiments, the antibody fragment comprises a heavy chain variable domain (e.g., V_{H}) and a light chain variable domain (e.g., a V_{L} or V_{K}). In certain embodiments, the antibody fragment comprises a Fab which comprises a heavy chain variable domain (e.g., V_{H}) and a light chain variable domain (e.g., a V_{L} or V_{K}).

In certain embodiments, the antibody fragment does not comprise any domains from the Fc region, e.g., does not comprise any CH2 or CH3 domains. In certain embodiments, the antibody fragment is an antibody fragment of, or derived from, any of a variety of antibodies, including, e.g., fully human, humanized or chimeric antibodies. The antibody from which the antibody fragment is obtained or derived can be of any of a variety of antibody classes, including, e.g., an IgG1 antibody, an IgG2 antibody, an IgG4 antibody. In some embodiments, the antibody fragment is obtained or derived from an agonistic antibody, e.g., an agonistic humanized antibody.

In embodiments where multiple types of fusion polypeptides having antibody fragments are used, the antibody fragments in the various types of fusion polypeptides may be capable of binding to different antigens, capable of binding the same epitope on an antigen, capable of binding to epitopes that are distinct and non-overlapping on an antigen, or capable of binding to epitopes that are distinct but overlapping on an antigen.

### 4. Linkers

In certain embodiments, linkers are used within fusion polypeptides and/or within single-chain molecules such as scFcs. In some embodiments, the linker is an amino acid linker. For example, a linker as employed herein may comprise from about 1 to about 100 amino acid residues, e.g., about 1 to about 70, about 2 to about 70, about 1 to about 30, or about 2 to about 30 amino acid residues. In some embodiments, the linker comprises at least 2, at least 3, at least 4, at least 5, at least 6, at least 7, at least 8, at least 9, or at least 10 amino acid residues.

In certain embodiments, the linker comprises a glycine-serine sequence, e.g., a (GₙS)ₘ sequence (e.g., GGS, GGGS (SEQ ID NO:18), or GGGGS (SEQ ID NO:19) sequence) that is present in at least 1, at least 2, at least 3, at least 4, at least 5, at least 6, at least 7, at least 8, at least 9, at least 10, at least 11, at least 12, at least 13, or at least 14 copies within the linker.

### B. Self-Assembled Polypeptide Complexes

In one aspect, provided are self-assembled polypeptide complexes comprising a plurality of fusion polypeptides as disclosed herein. Generally, provided self-assembled polypeptide complexes comprise (a) a plurality of first fusion polypeptides, each first fusion polypeptide comprising (1) an Fc polypeptide linked to (2) a nanocage monomer or subunit thereof, wherein the Fc polypeptide comprises an Fc chain having one or more mutations relative to a reference Fc chain of the same Ig class, and (b) a plurality of second fusion polypeptides, each second fusion polypeptide comprising (1) an antigen-binding antibody fragment linked to (2) a nanocage monomer or subunit thereof.

In some embodiments, the nanocage monomer is a ferritin monomer, and each fusion polypeptide within the self-assembled polypeptide complex comprises a ferritin light chain or a subunit of a ferritin light chain. In these embodiments, the self-assembled polypeptide complex does not comprise any ferritin heavy chains, subunits of ferritin heavy chains, or other ferritin components capable of binding to iron or capable of ferroxidase activity.

In some embodiments, the nanocage monomer or subunit thereof is a ferritin monomer subunit, and (a) each first fusion polypeptide comprises a ferritin monomer subunit which is C-half-ferritin and each second fusion polypeptide comprises a ferritin monomer subunit which is N-half-ferritin: or (b) each first fusion polypeptide comprises a ferritin monomer subunit which is N-half ferritin and each second fusion polypeptide comprises a ferritin monomer subunit which is C-half-ferritin.

In some embodiments, the self-assembled polypeptide complex comprises between 24 and 48 fusion polypeptides in total. In some embodiments, the self-assembled polypeptide complex comprises 24 fusion polypeptides in total. In some embodiments, the self-assembled polypeptide complex comprises more than 24 fusion polypeptides, e.g., at least 26, at least 28, at least 30, at least 32 fusion polypeptides, at least 34 fusion polypeptides, at least 36 fusion polypeptides, at least 38 fusion polypeptides, at least 40 fusion polypeptides, at least 42 fusion polypeptides, at least 44 fusion polypeptides, at least 46 fusion polypeptides, or at least 48 fusion polypeptides in total. In some embodiments, the self-assembled polypeptide complex comprises about 32 fusion polypeptides.

In some embodiments, the self-assembled polypeptide complex comprises at least 4, at least 5, least 6, at least 7, or at least 8 first fusion polypeptides.

In some embodiments, the self-assembled polypeptide complex comprises at least 4, at least 5, least 6, at least 7, or at least 8 second fusion polypeptides.

In some embodiments, the self-assembled polypeptide complex further comprises at least 4, at least 5, least 6, at least 7, at least 8, at least 9, at least 10, least 11, at least 12, at least 13, at least 14, at least 15, or at least 16 third fusion polypeptides.

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of first fusion polypeptides to all other fusion polypeptides.

### Pharmacokinetic Characteristics

In certain embodiments, when administered to a subject in need thereof, a provided self-assembled polypeptide complex, has one or more pharmacokinetic features similar to that of a reference IgG molecule (e.g., an IgG molecule whose class matches the class of an Fc chain within an Fc polypeptide of a first fusion polypeptide within the self-assembled polypeptide complex). In some embodiments, the ranges for the pharmacokinetic characteristics discussed herein (e.g., half-life, AUC, and/or Cₘₐₓ) are obtained when the self-assembled polypeptide complex is administered to a human subject. In some embodiments, the ranges for the pharmacokinetic characteristics discussed herein are obtained when the self-assembled polypeptide complex is administered via a systemic route, e.g., via intravenous or subcutaneous administration.

In some embodiments, a self-assembled polypeptide complex as disclosed herein has a similar half-life to that of reference IgG molecule. The reference IgG molecule may be, e.g., an antibody from which the antigen-binding antibody fragment within the second and/or third fusion polypeptide within the self-assembled polypeptide complex is derived. For example, if the antigen-binding fragment within the second and/or third fusion polypeptide comprises variable regions from “Antibody A,” then the reference IgG molecule may, in some embodiments, be “Antibody A.”

In some embodiments, after administration to a subject in need thereof, the self-assembled polypeptide complex has a half-life of between about 3 to 35 days, about 3 to about 28 days, about 3 to about 21 days, about 3 to about 14 days, about 3 to about 10 days, about 3 to about 7 days, about 3 to about 5 days, about 5 to about 35 days, about 5 to about 28 days, about 5 to about 21 days, about 5 to about 14 days, about 5 to about 10 days, about 5 to about 7 days, about 7 to about 35 days, about 7 to about 28 days, about 7 to about 21 days, about 7 to about 14 days, about 7 to about 10 days, about 10 to about 35 days, about 10 to about 28 days, about 10 to about 21 days, about 10 to about 14 days, about 14 to about 35 days, about 14 to about 28 days, about 14 to about 21 days, about 21 to about 35 days, or about 21 to about 28 days. In some embodiments, after administration to a subject in need thereof, the self-assembled polypeptide complex has a half-life of at least 3 days, at least 5 days, at least 7 days, at least 10 days, at least 14 days, at least 21 days, or at least 28 days. In some embodiments, after administration to a subject in need thereof, the self-assembled polypeptide complex is detectable in serum after at least 3 days, at least 5 days, at least 7 days, at least 10 days, at least 14 days, at least 21 days, or at least 28 days.

In some embodiments, a self-assembled polypeptide complex as disclosed herein has a similar bioavailability to that of reference IgG molecule, e.g., an antibody from which the Fab fragment comprised in the self-assembled polypeptide complex is derived. For example, in some embodiments, after administration to a subject in need thereof, the self-assembled polypeptide complex has an area-under-the-curve (AUC) of between about 10 to about 8000 day·μg/mL, about 10 to about 7000 day·μg/mL, about 10 to about 6000 day·μg/mL, about 10 to about 5000 day·μg/mL, about 10 to about 4000 day·μg/mL, about 10 to about 3000 day·μg/mL, about 10 to about 2500 day·μg/mL, about 10 to about 1000 day·μg/mL, about 10 to about 1500 day·μg/mL, about 10 to about 1000 day·μg/mL, about 10 to about 750 day·μg/mL, about 10 to about 500 day·μg/mL, about 10 to about 400 day·μg/mL, about 10 to about 300 day·μg/mL, about 10 to about 200 day·μg/mL, about 10 to about 100 day·μg/mL, about 10 to about 50 day·μg/mL, about 10 to about 25 day·μg/mL, about 25 to about 8000 day·μg/mL, about 25 to about 7000 day·μg/mL, about 25 to about 6000 day·μg/mL, about 25 to about 5000 day·μg/mL, about 25 to about 4000 day·μg/mL, about 25 to about 3000 day·μg/mL, about 25 to about 2500 day·μg/mL, about 25 to about 1000 day·μg/mL, about 25 to about 1500 day·μg/mL, about 25 to about 1000 day·μg/mL, about 25 to about 750 day·μg/mL, about 25 to about 500 day·μg/mL, about 25 to about 400 day·μg/mL, about 25 to about 300 day·μg/mL, about 25 to about 200 day·μg/mL, about 25 to about 100 day·μg/mL, about 25 to about 50 day·μg/mL, about 50 to about 8000 day·μg/mL, about 50 to about 7000 day·μg/mL, about 50 to about 6000 day·μg/mL, about 50 to about 5000 day·μg/mL, about 50 to about 4000 day·μg/mL, about 50 to about 3000 day·μg/mL, about 50 to about 2500 day·μg/mL, about 50 to about 2000 day·μg/mL, about 50 to about 1500 day·μg/mL, about 50 to about 1000 day·μg/mL, about 50 to about 750 day·μg/mL, about 50) to about 500 day·μg/mL, about 50) to about 400 day·μg/mL, about 50 to about 300 day·μg/mL, about 50) to about 200 day·μg/mL, about 50 to about 100 day·μg/mL, about 100 to about 8000 day·μg/mL, about 100 to about 7000 day·μg/mL, about 100 to about 6000 day·μg/mL, about 100 to about 5000 day·μg/mL, about 100 to about 4000 day·μg/mL, about 100 to about 3000 day·μg/mL, about 100 to about 2500 day·μg/mL, about 100 to about 1000 day: μg/mL, about 100 to about 1500 day·μg/mL, about 100 to about 1000 day·μg/mL, about 100 to about 750 day·μg/mL, about 100 to about 500 day·μg/mL, about 100 to about 400 day·μg/mL, about 100 to about 300 day·μg/mL, about 100 to about 200 day·μg/mL, about 200 to about 8000 day·μg/mL, about 200 to about 7000 day·μg/mL, about 200 to about 6000 day·μg/mL, about 200 to about 5000 day·μg/mL, about 200 to about 4000 day·μg/mL, about 200 to about 3000 day·μg/mL, about 200 to about 2000 day·μg/mL, about 200 to about 1000 day·μg/mL, about 200 to about 1500 day·μg/mL, about 200 to about 1000 day·μg/mL, about 200 to about 750 day·μg/mL, about 200 to about 500 day·μg/mL, about 200 to about 400 day·μg/mL, about 200 to about 300 day·μg/mL, about 300 to about 8000 day·μg/mL, about 300 to about 7000 day·μg/mL, about 300 to about 6000 day·μg/mL, about 300 to about 5000 day·μg/mL, about 300 to about 4000 day·μg/mL, about 300 to about 3000 day·μg/mL, about 300 to about 2500 day·μg/mL, about 300 to about 2000 day·μg/mL, about 300 to about 1500 day·μg/mL, about 300 to about 1000 day·μg/mL, about 300 to about 750 day·μg/mL, about 300 to about 500 day·μg/mL, about 300 to about 400 day·μg/mL, about 400 to about 8000 day·μg/mL, about 400) to about 7000 day·μg/mL, about 400 to about 6000 day·μg/mL, about 400 to about 5000 day·μg/mL, about 400 to about 4000 day·μg/mL, about 400 to about 3000 day·μg/mL, about 400 to about 2500 day·μg/mL, about 400 to about 2000 day·μg/mL, about 400 to about 1500 day·μg/mL, about 400 to about 1000 day·μg/mL, about 400 to about 750 day·μg/mL, about 400 to about 500 day·μg/mL, about 500 to about 8000 day·μg/mL, about 500 to about 7000 day·μg/mL, about 500 to about 6000 day·μg/mL, about 500 to about 5000 day·μg/mL, about 500 to about 4000 day·μg/mL, about 500 to about 3000 day·μg/mL, about 500 to about 2500 day·μg/mL, about 500 to about 2000 day·μg/mL, about 500 to about 1500 day·μg/mL, about 500 to about 1000 day·μg/mL, about 500 to about 750 day·μg/mL, about 750 to about 8000 day·μg/mL, about 750 to about 7000 day·μg/mL, about 750 to about 6000 day·μg/mL, about 750 to about 5000 day·μg/mL, about 750 to about 4000 day·μg/mL, about 750 to about 3000 day·μg/mL, about 750 to about 2500 day·μg/mL, about 750 to about 2000 day·μg/mL, about 750 to about 1500 day·μg/mL, about 750 to about 1000 day·μg/mL, about 1000 to about 8000 day·μg/mL, about 1000 to about 7000 day·μg/mL, about 1000 to about 6000 day·μg/mL, about 1000 to about 5000 day·μg/mL, about 1000 to about 4000 day·μg/mL, about 1000 to about 3000 day·μg/mL, about 1000 to about 2500 day·μg/mL, about 1000 to about 2000 day·μg/mL, about 1000 to about 1500 day·μg/mL, about 1500 to about 8000 day·μg/mL, about 1500 to about 7000 day·μg/mL, about 1500 to about 6000 day·μg/mL, about 1500 to about 5000 day·μg/mL, about 1500 to about 4000 day·μg/mL, about 1500 to about 3000 day·μg/mL, about 1500 to about 2500 day·μg/mL, about 1500 to about 2000 day·μg/mL, about 2000 to about 8000 day·μg/mL, about 2000 to about 7000 day·μg/mL, about 2000 to about 6000 day·μg/mL, about 2000 to about 5000 day·μg/mL, about 2000 to about 4000 day·μg/mL, about 2000 to about 3000 day·μg/mL, about 2000 to about 2500 day·μg/mL, about 2500 to about 8000 day·μg/mL, about 2500 to about 7000 day·μg/mL, about 2500 to about 6000 day·μg/mL, about 2500 to about 5000 day·μg/mL, about 2500 to about 4000 day·μg/mL, about 2500 to about 3000 day·μg/mL, about 3000 to about 8000 day·μg/mL, about 3000 to about 7000 day·μg/mL, about 3000 to about 6000 day·μg/mL, about 3000 to about 5000 day·μg/mL, about 3000 to about 4000 day·μg/mL, about 4000 to about 8000 day·μg/mL, about 4000 to about 7000 day·μg/mL, about 4000 to about 6000 day·μg/mL, about 4000 to about 5000 day·μg/mL, about 5000 to about 8000 day: μg/mL, about 5000 to about 7000 day·μg/mL, about 5000 to about 6000 day·μg/mL, about 6000 to about 8000 day·μg/mL, about 6000 to about 7000 day·μg/mL, or about 7000 to about 8000 day·μg/mL. In some embodiments, after administration to a subject in need thereof, the self-assembled polypeptide complex has an AUC of at least 10 day·μg/mL, at least 25 day·μg/mL, at least 50 day·μg/mL, at least 100 day·μg/mL, at least 200 day·μg/mL, at least 300 day·μg/mL, at least 400 day·μg/mL, at least 500 day·μg/mL, at least 750 day·μg/mL, at least 1000 day·μg/mL, at least 1500 day·μg/mL, at least 2000 day·μg/mL, at least 2500 day·μg/mL, at least 3000 day·μg/mL, at least 4000 day·μg/mL, at least 5000 day·μg/mL, at least 6000 day·μg/mL, at least 7000 day·μg/mL, or at least 8000 day·μg/mL.

In some embodiments, a self-assembled polypeptide complex as disclosed herein has a similar bioavailability to that of reference IgG molecule. For example, in some embodiments, after administration to a subject in need thereof, the self-assembled polypeptide complex has a maximum concentration (Cₘₐₓ) of between about 10 μg/mL to about 750 mg/mL, about 25 μg/mL to about 750 mg/mL, about 50 μg/mL to about 750) mg/mL, about 75 μg/mL to about 750 mg/mL, about 100 μg/mL to about 750 mg/mL, about 250) μg/mL to about 750) mg/mL, about 500 μg/mL to about 750 mg/mL, about 750) μg/mL to about 750) mg/mL, about 1 mg/mL to about 750) mg/mL, about 10 mg/mL to about 750) mg/mL, about 25 mg/mL to about 750) mg/mL, about 50 mg/mL to about 750) mg/mL, about 75 mg/mL to about 750 mg/mL, about 100 mg/mL to about 750) mg/mL, about 250) mg/mL to about 750) mg/mL, about 500 mg/mL to about 750) mg/mL, about 10) μg/mL to about 500) mg/mL, about 25 μg/mL to about 500 mg/mL, about 50 g/mL to about 500 mg/mL, about 75 μg/mL to about 500 mg/mL, about 100 μg/mL to about 500 mg/mL, about 250 μg/mL to about 500 mg/mL, about 500 μg/mL to about 500 mg/mL, about 750) μg/mL to about 500 mg/mL, about 1 mg/mL to about 500 mg/mL, about 10 mg/mL to about 500 mg/mL, about 25 mg/mL to about 500 mg/mL, about 50 mg/mL to about 500 mg/mL, about 75 mg/mL to about 500 mg/mL, about 100 mg/mL to about 500 mg/mL, about 250) mg/mL to about 500 mg/mL, about 10) μg/mL to about 250 mg/mL, about 25 μg/mL to about 250 mg/mL, about 50 μg/mL to about 250 mg/mL, about 75 μg/mL to about 250 mg/mL, about 100 μg/mL to about 250 mg/mL, about 250) μg/mL to about 250 mg/mL, about 500 μg/mL to about 250) mg/mL, about 750) μg/mL to about 250 mg/mL, about 1 mg/mL to about 250 mg/mL, about 10 mg/mL to about 250) mg/mL, about 25 mg/mL to about 250) mg/mL, about 50 mg/mL to about 250 mg/mL, about 75 mg/mL to about 250 mg/mL, about 100 mg/mL to about 250 mg/mL, about 10 μg/mL to about 100 mg/mL, about 25 μg/mL to about 100 mg/mL, about 50 μg/mL to about 100 mg/mL, about 75 μg/mL to about 100 mg/mL, about 100 μg/mL to about 100 mg/mL, about 250) μg/mL to about 100 mg/mL, about 500 μg/mL to about 100 mg/mL, about 750) μg/mL to about 100 mg/mL, about 1 mg/mL to about 100 mg/mL, about 10 mg/mL to about 100 mg/mL, about 25 mg/mL to about 100 mg/mL, about 50 mg/mL to about 100 mg/mL, about 75 mg/mL to about 100 mg/mL, about 10 μg/mL to about 75 mg/mL, about 25 μg/mL to about 75 mg/mL, about 50 μg/mL to about 75 mg/mL, about 75 μg/mL to about 75 mg/mL, about 100 μg/mL to about 75 mg/mL, about 250 μg/mL to about 75 mg/mL, about 500 μg/mL to about 75 mg/mL, about 750) μg/mL to about 75 mg/mL, about 1 mg/mL to about 75 mg/mL, about 10 mg/mL to about 75 mg/mL, about 25 mg/mL to about 75 mg/mL, about 50 mg/mL to about 75 mg/mL, about 10 μg/mL to about 50 mg/mL, about 25 μg/mL to about 50 mg/mL, about 50 μg/mL to about 50 mg/mL, about 75 μg/mL to about 50 mg/mL, about 100 μg/mL to about 50 mg/mL, about 250 μg/mL to about 50 mg/mL, about 500 μg/mL to about 50 mg/mL, about 750 μg/mL to about 50 mg/mL, about 1 mg/mL to about 50 mg/mL, about 10 mg/mL to about 50 mg/mL, about 25 mg/mL to about 50 mg/mL, about 10 μg/mL to about 25 mg/mL, about 25 μg/mL to about 25 mg/mL, about 50 g/mL to about 25 mg/mL, about 75 μg/mL to about 25 mg/mL, about 100 μg/mL to about 25 mg/mL, about 250 μg/mL to about 25 mg/mL, about 500 μg/mL to about 25 mg/mL, about 750) μg/mL to about 25 mg/mL, about 1 mg/mL to about 25 mg/mL, about 10 mg/mL to about 25 mg/mL, about 10 μg/mL to about 10 mg/mL, about 25 μg/mL to about 10 mg/mL, about 50 g/mL to about 10 mg/mL, about 75 μg/mL to about 10 mg/mL, about 100 μg/mL to about 10 mg/mL, about 250 μg/mL to about 10 mg/mL, about 500 μg/mL to about 10 mg/mL, about 750 μg/mL to about 10 mg/mL, about 1 mg/mL to about 10 mg/mL, about 10 μg/mL to about 1 mg/mL, about 25 μg/mL to about 1 mg/mL, about 50 μg/mL to about 1 mg/mL, about 75 μg/mL to about 1 mg/mL, about 100 μg/mL to about 1 mg/mL, about 250 μg/mL to about 1 mg/mL, about 500 μg/mL to about 1 mg/mL, about 750 μg/mL to about 1 mg/mL, about 10 μg/mL to about 750 μg/mL, about 25 μg/mL to about 750 μg/mL, about 50 μg/mL to about 750 μg/mL, about 75 μg/mL to about 750 μg/mL, about 100 μg/mL to about 750 μg/mL, about 250 μg/mL to about 750 μg/mL, about 500 μg/mL to about 750 μg/mL, about 10 μg/mL to about 500 μg/mL, about 25 μg/mL to about 500 μg/mL, about 50 μg/mL to about 500 μg/mL, about 75 μg/mL to about 500 μg/mL, about 100 μg/mL to about 500 μg/mL, about 250 μg/mL to about 500 μg/mL, about 10 μg/mL to about 250 μg/mL, about 25 μg/mL to about 250 μg/mL, about 50 μg/mL to about 250 μg/mL, about 75 μg/mL to about 250 μg/mL, about 100 μg/mL to about 250 μg/mL, about 10 μg/mL to about 100 μg/mL, about 25 μg/mL to about 100 μg/mL, about 50 g/mL to about 100 μg/mL, about 75 μg/mL to about 100 μg/mL, about 10 μg/mL to about 75 μg/mL, about 25 μg/mL to about 75 μg/mL, about 50 μg/mL to about 75 μg/mL, about 10 μg/mL to about 50 μg/mL, about 25 μg/mL to about 50 μg/mL, or about 10 μg/mL to about 25 μg/mL. In some embodiments, after administration to a subject in need thereof, the self-assembled polypeptide complex has a maximum concentration (Cₘₐₓ) of at least 10 μg/mL, at least 25 μg/mL, at least 50 μg/mL, at least 100 μg/mL, at least 250 μg/mL, at least 500 μg/mL, at least 750 μg/mL, at least 1 mg/mL, at least 10 mg/mL, at least 25 mg/mL, at least 50 mg/mL, at least 75 mg/mL, at least 100 mg/mL, at least 250 mg/mL, at least 500 mg/mL, or at least 750 mg/mL when administered to a subject in need thereof.

### Effects

In some embodiments, administration of a self-assembled polypeptide complex as disclosed herein to a subjectresults in an improvement in a clinical outcome or metric in the subject. For example, in a subject with a tumor, administration of the self-assembled polypeptide complex may inhibit or slow progression of the tumor, e.g., cause regression of the tumor. In some embodiments administration of the self-assembled polypeptide complex results in complete regression of the tumor.

### C. Methods of Treatment

In one aspect, provided are methods that may be useful for treating, ameliorating, or preventing a disease or a condition (e.g., an infectious disease, cancer, or an autoimmune disease), generally comprising a step of administering a composition comprising a self-assembled polypeptide complex of the present disclosure to a subject.

In some embodiments, the subject is a mammal, e.g., a human.

Compositions for administration to subjects generally comprise a self-assembled polypeptide complex as disclosed herein. In some embodiments, such compositions further comprise a pharmaceutically acceptable excipient.

Compositions may be formulated for administration for any of a variety of routes of administration, including systemic routes (e.g., oral, inhalation, intranasal, intravenous, intraperitoneal, subcutaneous, or intramuscular administration).

In some embodiments, the step of administering results in improvement in one or more clinical outcomes or metrics in the subject.

In some embodiments, the step of administering results in slowing or inhibiting progression of the tumor, e.g., regression of the tumor. In some embodiments, the step of administering results in complete regression of the tumor.

### EXAMPLES

### Example 1. Construction and Expression of Representative DR5-Targeting Multabodies

This Example describes the generation of DR5-targeting Multabodies (MBs) comprising a combination of fusion proteins comprising a (1) human ferritin light chain or subunit thereof and (2) a single-chain Fab (scFab) or a single chain Fc-dimer (scFc), fused via a linker, such as a (Glyn-Ser) m amino acid linker as described herein.

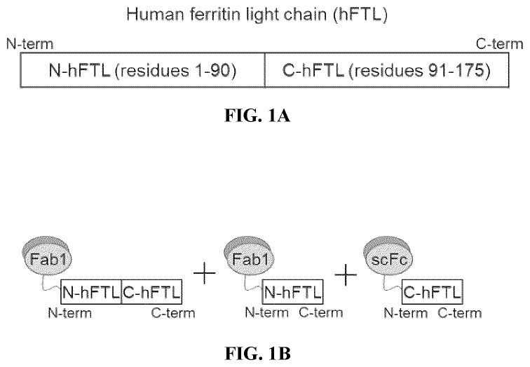

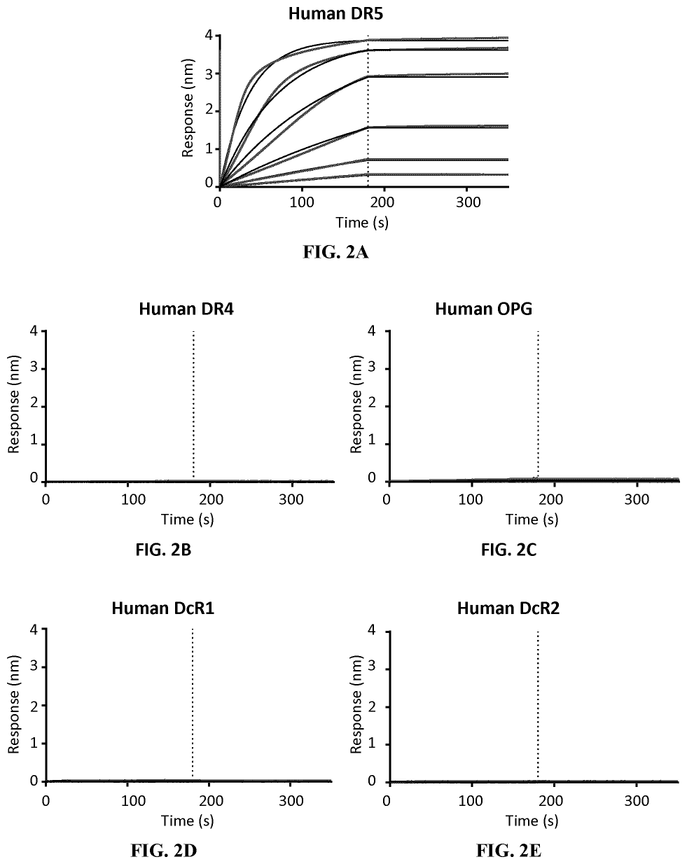

Genes encoding fusion proteins (1) scFab of an anti-DR5 antibody (in this case, conatumumab) fused to the N-terminus of hFTL (aDR5-hFTL, SEQ ID NO:9), (2) scFab of an anti-DR5 antibody fused to the N-terminus of an N-half ferritin (aDR5-N_hFTL, SEQ ID NO: 11), and (3) scFc fused to the N-terminus of a C-half ferritin (various scFc-C_hFTL constructs, as described further below) were prepared, mixed at a molar ratio of 2:1:1 and transiently transfected into ExpiCHO-S cells for the production and formation of the DR5-targeting MBs. See FIG. 1B.

The scFc-ferritin fusion proteins comprised wild-type (WT) or engineered IgG1 Fc chains. The engineered IgG1 Fc chains contained various combinations of the L234A, L235A, G236R, G237A, P329G, and A330L mutations, as shown in Table 1 below. Numbering in Table 1 is according to the EU numbering scheme.

TABLE 1
Residues at certain positions within IgG1 Fc chains used in Multabodies
and corresponding SEQ ID NOs of scFc-C hFTL constructs

| | | | | | | | SEQ ID NO: |
|---|---|---|---|---|---|---|---|
| | Amino acid position | | | | | | of corresponding |
| | 234 | 235 | 236 | 237 | 329 | 330 | scFc-C_hFTL construct |
| WT | L | L | G | G | P | A | SEQ ID NO: 13 |
| (wild type) | | | | | | | |
| LALA | A | A | | | | | SEQ ID NO: 14 |
| LALAP | A | A | | | G | | SEQ ID NO: 15 |
| LLRAL | A | A | R | A | | L | SEQ ID NO: 16 |
| LLGRAL | A | A | R | A | G | L | SEQ ID NO: 17 |

In rows corresponding to sets of engineered Fc chains, residues are only shown at positions that differ from the wild type residue.

### Example 2. Target Binding of Multabodies Determined by Biolayer Interferometry

The binding kinetics and affinity of exemplary MBs generated as described in Example 1 to recombinant human DR5 (hDR5), cynomolgus DR5 (cDR5), mouse DR5 (mDR5), and rat DR5 (rDR5) were determined by biolayer interferometry (BLI) using an Octet RED96 instrument. Binding characteristics were determined for (1) conatumumab, a fully human monoclonal IgG1 antibody that binds to DR5, and (2) Cona MB IgG1 LLRAL, a Multabody that includes fusion polypeptides that include a scFc with the “LLRAL” mutations described in Table 1, and which also includes fusion polypeptides that comprise a scFab derived from conatumumab.

Briefly, Ni-NTA biosensors were coated with hDR5-His, cDR5-His, mDR5-His, or rDR5-His (extracellular domain of hDR5, cDR5, mDR5, or rDR5 with a C-terminal polyhistidine tag) to reach a signal response of 0.8 nm. The coated biosensors were dipped into wells containing serial dilutions of the test MBs (20-10-5-2.5-1.25-0.63 nM) in PBS-0.02% T-0.01% BSA (PBS supplemented with 0.02% (v/v) Tween 20 and 0.01% (w/v) BSA) for 180 s (association phase) and then into PBS-0.02% T-0.01% BSA for 180 s (dissociation phase). All measurements were performed at 30° C. in PBS-0.02% T-0.01% BSA, pH 7.4, with shaking speed 1000 rpm and monitored in real time. Biosensors were regenerated between experiments by applying 10 mM glycine, pH 1.7, for 5 s for four times, followed by recharging with 10 mM NiSO₄ for 1 min.

Target binding was evaluated based on the maximal association binding response at the end of the association phase, the dissociation rate (k_{off}), and/or the equilibrium dissociation constant (K_{D}) calculated using a 1:1 fitting model. A maximal association binding response of less than 0.1 nm when test MB at 20 nM was classified as “non-binding.”

The values determined for kₒₙ, k_{off}, and the resulting K_{D} for the test molecules are summarized in Table 2. Cona MB IgG1 LLRAL bound to human and cynomolgus DR5 with approximately picomolar affinity or less and showed no binding to mouse or rat DR5. (The instrument has detection limit of 1 picomolor.) These observations are consistent with conatumumab's (“Cona”) binding profile.

TABLE 2
Kinetic constants and affinities to target binding of Multabodies determined by BLI

| | hDR5 | | | cDR5 | | | | |
|---|---|---|---|---|---|---|---|---|
| | kₒₙ | k_{off} | K_{D} | kₒₙ | k_{off} | K_{D} | | |
| Test article | [M⁻¹ × s⁻¹] | [s⁻¹] | [M] | M⁻¹ × s⁻¹] | [s⁻¹] | [M] | mDR5 | rDR5 |
| Cona MB | 1.4 × 10⁶ | <1.0 × 10⁻⁷ | <1.0 × 10⁻¹² | 1.5 × 10⁶ | <1.0 × 10⁻⁷ | <1.0 × 10⁻¹² | Non- | Non- |
| IgG1 LLRAL | | | | | | | binding | binding |
| Cona | 2.4 × 10⁵ | 2.2 × 10⁻⁵ | 8.9 × 10⁻¹¹ | ND | ND | ND | | |

### Example 3. Binding Specificity of Multabodies Determined by Biolayer Interferometry

BLI was employed to assess the non-specific binding of exemplary DR-targeting MBs to related tumor necrosis factor receptor superfamily (TNFRSF) members.

The experiments were performed similarly as described in Example 2, except that His-tagged extracellular domains of human DR4, human osteoprotegerin (OPG), human decoy receptor 1 (DcR1), or human DcR2-hDR4-His, hOPG-His, hDcR1-His, or hDcR2-His were coated onto Ni-NTA biosensors and titrated with test MBs at various concentrations.

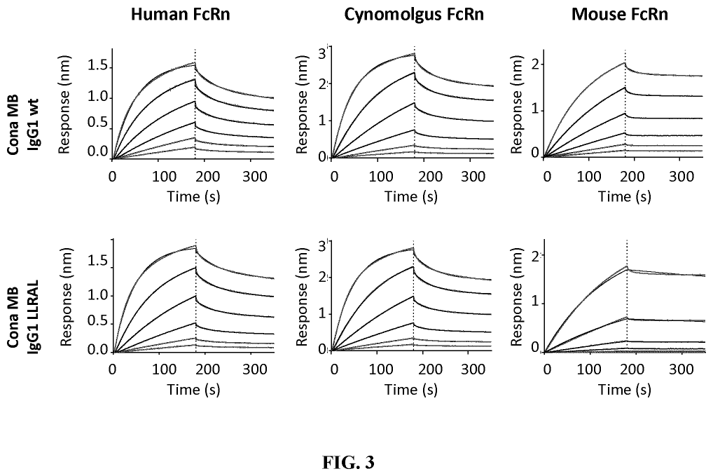

FIGS. 2A, 2B, 2C, 2D, and 2E show representative examples of relevant segments of the resulting sensorgrams. Cona MB IgG1 LLRAL bound DR5 with high specificity and showed no binding to other tested TNFRSF members.

### Example 4. Binding of Multabodies to Fc Receptors Determined by Biolayer Interferometry

The binding kinetics and affinities of various DR5-targeting MBs (each comprising different sets of Fc mutations and generated as described in Example 1) to various Fc receptors were determined by BLI.

All MBs tested in this Example contained polypeptides comprising scFabs derived from conatumumab, which binds to DR5. These DR5-targeting MBs (“Cona MB”) also included polypeptides having scFcs with the either wild type Fc chains (IgG1 wt) or Fc chains with a certain combination of mutations (IgG1 LLRAL). (See Table 1)

Binding to the following human, cynomolgus monkey, and mouse Fc receptors were determined: human Fc gamma receptor type I (hFcγRI), hFcγRIIa, hFcγRIIb, hFcγRIIIa, hFcγRIIIb, human neonatal Fc receptor (hFcRn), cynomolgus monkey FcγRI (cFcγRI), cFcγRIIa, cFcγRIIb, cFcγRIII, cFcRn, mouse FcγRI (mFcγRI), mFcγRIIb, mFcγRIII, mFcγRIV, and mFcRn.

Experiments were performed similarly as described in Example 2, except that His-tagged Fc receptors were coated onto Ni-NTA biosensors and titrated with test MBs at various concentrations. To assess the potential of the MBs to undergo endosomal recycling, binding to FcRn was measured at pH 6.0 for association and pH 7.4 for dissociation.

Table 3 summarizes the kₒₙ-k_{off}, and K_{D} values determined for the MBs binding to various FcγRs. Cona MB with wild type IgG1 Fc (IgG1 WT) bound to human, cynomolgus monkey, and mouse FcγRs. Cona MB with IgG1 LLRAL showed diminished binding to human, cynomolgus monkey, and mouse FcγRs.

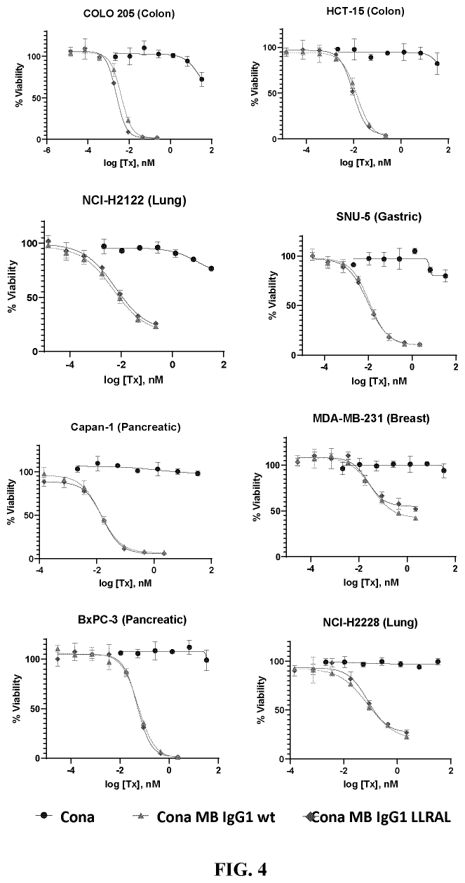

FIG. 3 shows representative examples of relevant segments of resulting sensorgrams from the FcRn binding studies. All tested MBs bound to human, cynomolgus monkey, and mouse FcRns at pH 6.0 and dissociated from the receptors at pH 7.4. Cona MB IgG1 LLRAL dissociated from human or cynomolgus monkey FcRn at pH 7.4 at a comparable rate to Cona MB IgG1 WT.

TABLE 3
Kinetic constants and affinities to FcγRs
of Multabodies determined by BLI

| | | Cona MB | Cona MB |
|---|---|---|---|
| | | IgG1 WT | IgG1 LLRAL |
| hFcγRI | kₒₙ [M⁻¹ × s⁻¹] | 5.67 × 10⁵ | Non-binding |
| | k_{off} [s⁻¹] | <1.0 × 10⁻⁷ | |
| | K_{D} [M] | <1.0 × 10⁻¹² | |
| hFcγRIIa | kₒₙ [M⁻¹ × s⁻¹] | 1.34 × 10⁶ | Non-binding |
| | k_{off} [s⁻¹] | <1.0 × 10⁻⁷ | |
| | K_{D} [M] | <1.0 × 10⁻¹² | |
| hFcγRIIb | kₒₙ [M⁻¹ × s⁻¹] | 1.47 × 10⁶ | Non-binding |
| | k_{off} [s⁻¹] | <1.0 × 10⁻⁷ | |
| | K_{D} [M] | 4.81 × 10⁻¹² | |
| hFcγRIIIa | kₒₙ [M⁻¹ × s⁻¹] | 1.32 × 10⁶ | Non-binding |
| | k_{off} [s⁻¹] | <1.0 × 10⁻⁷ | |
| | K_{D} [M] | <1.0 × 10⁻¹² | |
| hFcγRIIIb | kₒₙ [M⁻¹ × s⁻¹] | 1.57 × 10⁶ | Non-binding |
| | k_{off} [s⁻¹] | 8.44 × 10⁻⁵ | |
| | K_{D} [M] | 5.37 × 10⁻¹¹ | |
| cFcγRI | kₒₙ [M⁻¹ × s⁻¹] | 4.72 × 10⁵ | Non-binding |
| | k_{off} [s⁻¹] | <1.0 × 10⁻⁷ | |
| | K_{D} [M] | <1.0 × 10⁻¹² | |
| cFcγRIIa | kₒₙ [M⁻¹ × s⁻¹] | 1.01 × 10⁶ | Non-binding |
| | k_{off} [s⁻¹] | 2.01 ×10⁻⁴ | |
| | K_{D} [M] | 1.99 × 10⁻¹⁰ | |
| cFcγRIIb | kₒₙ [M⁻¹ × s⁻¹] | 1.07 × 10⁶ | Non-binding |
| | k_{off} [s⁻¹] | 1.79 × 10⁻⁴ | |
| | K_{D} [M] | 1.67 × 10⁻¹⁰ | |
| cFcγRIII | kₒₙ [M⁻¹ × s⁻¹] | 3.22 × 10⁵ | Non-binding |
| | k_{off} [s⁻¹] | <1.0 × 10⁻⁷ | |
| | K_{D} [M] | <1.0 × 10⁻¹² | |
| mFcγRI | kₒₙ [M⁻¹ × s⁻¹] | 7.46 × 10⁵ | Non-binding |
| | k_{off} [s⁻¹] | <1.0 × 10⁻⁷ | |
| | K_{D} [M] | <1.0 × 10⁻¹² | |
| mFcγRIIb | kₒₙ [M⁻¹ × s⁻¹] | 8.89 × 10⁵ | Non-binding |
| | k_{off} [s⁻¹] | 2.98 × 10⁻⁴ | |
| | K_{D} [M] | 3.35 × 10⁻¹⁰ | |
| mFcγRIII | kₒₙ [M⁻¹ × s⁻¹] | 7.04 × 10⁵ | Non-binding |
| | k_{off} [s⁻¹] | 4.90 × 10⁻⁴ | |
| | K_{D} [M] | 6.96 × 10⁻¹⁰ | |
| mFcγRIV | kₒₙ [M⁻¹ × s⁻¹] | 3.56 × 10⁵ | Non-binding |
| | k_{off} [s⁻¹] | <1.0 × 10⁻⁷ | |
| | K_{D} [M] | <1.0 × 10⁻¹² | |

### Example 5. Assessment of In Vitro Tumor Cell Cytotoxicity

The cytotoxicity of exemplary DR5-targeting MBs (generated as described in Example 1) was evaluated using different tumor cell lines. Cona MB IgG1 wt and Cona MB IgG1 LLRAL were tested on COLO 205 (human colon carcinoma), HCT-15 (human colon carcinoma), NCI-H2122 (human lung carcinoma), SNU-5 (human gastric carcinoma), Capan-1 (human pancreatic carcinoma), MDA-MB-231 (invasive ductal carcinoma), BxPC-3 (human pancreatic carcinoma), and NCI-H2228 (human lung carcinoma) cell lines.

Tumor cell lines were seeded at 5000 cells per well in a 96-well plate and incubated overnight to facilitate attachment. The next day, cells were treated with serial dilutions of DR5-targeting MBs or anti-DR5 (conatumumab) and incubated for 24 h at 37° C. Cell viability was measured using the CellTiter-Glo Luminescence Cell Viability Assay (Promega), according to the manufacturer's instruction. Briefly, assay reagent (100 μL) was added to 100 μL cells at room temperature, mixed using a plate shaker at 500 rpm for 2 min, and incubated at room temperature for 10 min. Luminescent signals were read using the Synergy Neo2 Multi-Mode Assay Microplate Reader (BioTek Instruments).

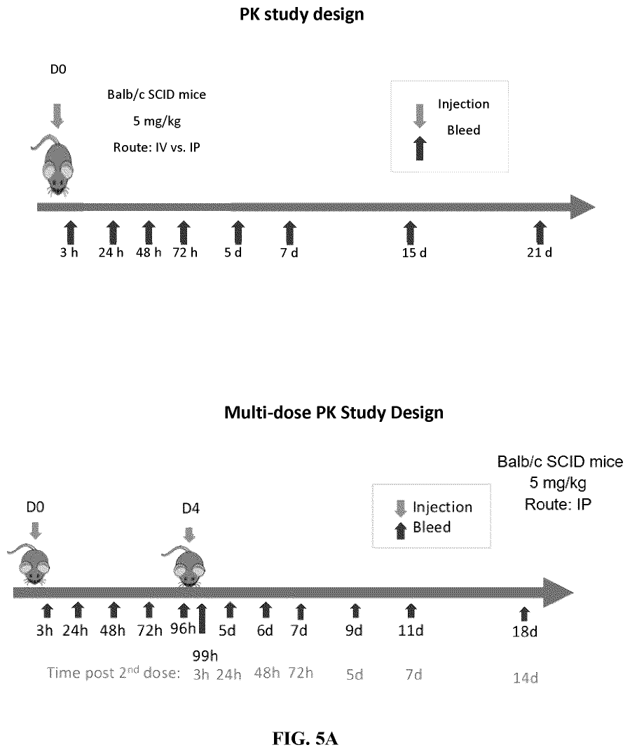

FIG. 4 shows results from these experiments. In FIG. 4, the percentage of viable cells (compared to vehicle-treated tumor cells) was plotted on the y-axis versus the test molecule concentration on the x-axis. The data were fitted using Prism 9.1.2 software (GraphPad) with nonlinear regression (log inhibitor vs. response, variable slope, 4 parameters). Table 4 provides the resulting IC₅₀ values. Both of the tested DR5-targeting MBs (Cona MB IgG1 wt and Cona MB IgG1 LLRAL) were capable of inducing cytotoxicity of human cancer cells lines. On the other hand, conatumumab—an IgG1 antibody—did not induce, or very poorly induced, tumor cell cytotoxicity.

TABLE 4
Assessment of tumor cell cytotoxicity

| | | | Cona MB | Cona MB |
|---|---|---|---|---|
| | | Cona | IgG1 wt | IgG1 LLRAL |
| IC₅₀ | COLO 205 | >33000 | 4.06 | 2.38 |
| [pM] | HCT-15 | >33000 | 13.58 | 9.53 |
| | NCI-2122 | >33000 | 4.99 | 5.86 |
| | SNU-5 | >33000 | 11.14 | 9.51 |
| | Capan-1 | >33000 | 13.73 | 15.7 |
| | MDA-MB-231 | >33000 | 34.17 | 23.96 |
| | BxPC-3 | >33000 | 54.64 | 50.2 |
| | NCI-H2228 | >33000 | 79.67 | 77.15 |

### Example 6. Pharmacokinetics of Multabodies in Mice

The pharmacokinetics (PK) of exemplary DR5-targeting MBs generated as described in Example 1 (Cona MB IgG1 LLRAL and conatumumab) were analyzed in 8-week old female BALB/c SCID mice (Jackson Labs).

For single-dose PK studies, mice received a single bolus injection of 200 μL test molecule at 5 mg/kg intraperitoneally (i.p.) or intravenously (i.v.) (N=8 mice per group, N=4 mice per timepoint). 50-100 μL blood samples were collected at the timepoints of 3 h, 24 h, 48 h, 72 h, 5 days, 7 days, 15 days, and 21 days.

For multi-dose PK studies, mice were injected with 200 μL test molecule at 5 mg/kg on day 9, followed by a second injection of 200 μL test molecule at 5 mg/kg 96 h after. 50-100 μL blood samples were collected 3 h, 24 h, 48 h, 72 h, and 96 h after the first dose and 3 h, 24 h, 48 h, 72 h, 5 days, 7 days, and 14 days after the second dose.

Blood samples were collected from the saphenous vein into heparin-coated tubes and finger-vortexed to ensure mixing. Subsequently, the samples were centrifuged at 1500× g at 4° C. for 15 min, and the plasma samples were collected and immediately stored at −80° C. until use in ELISA assay.

To measure plasma drug level with ELISA, recombinant DR5 at 2 μg/mL in PBS was coated onto Maxisorp plates (Fisher Scientific) overnight at 4° C. After washing twice with PBS-0.05% T (PBS supplemented with 0.05% (v/v) Tween-20), the plate was blocked with 3% (w/v) BSA in PBS for 1 h at room temperature and subsequently washed twice with PBS-0.05% T. Plasma samples were diluted in PBS-0.05% T-0.5% BSA (PBS supplemented with 0.05% (v/v) Tween-20 and 0.5% (w/v) BSA), added to wells, and incubated for 1 h at room temperature and a shaking frequency of 500 rpm, followed by another wash step with PBS-0.05% T. Bound molecules were detected by incubation with 1:10000 diluted goat polyclonal anti-human Fc-HRP secondary antibody (Jackson Immunoresearch). After a further wash step with PBS-0.05% T, OptEIA™ TMB Substrate Reagent Set (BD Biosciences) was used for detection following the manufacturer's instruction, and the absorbances at 450 nm were read using the Synergy Neo2 Multi-Mode Assay Microplate Reader (BioTek Instruments). A calibration curve was prepared using the dilutions of test molecule in PBS-0.05% T-0.5% BSA.

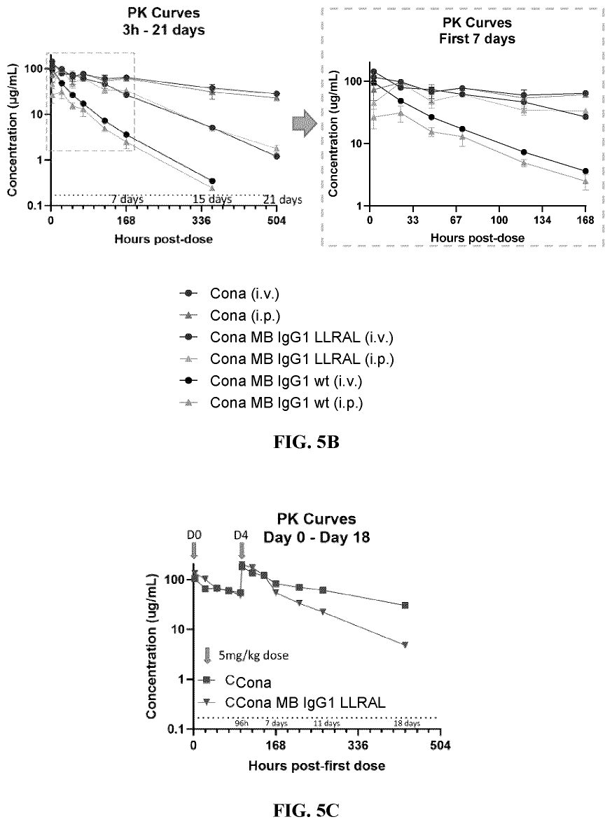

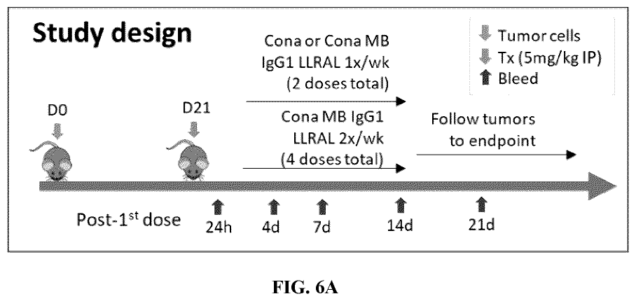

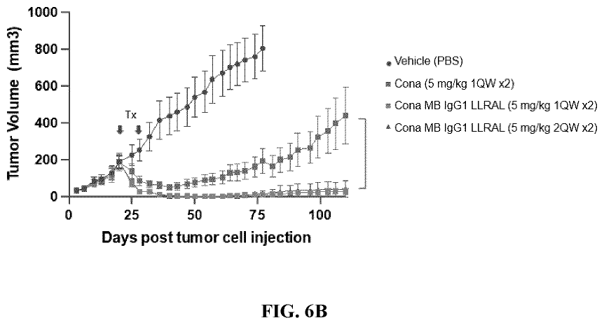

FIG. 5A is a schematic illustrating the design of these pharmacokinetic studies.

FIG. 5B shows plots of the plasma concentration over time in a single-dose PK study for Cona MB IgG1 wt, Cona MB IgG1 LLRAL, and conatumumab (“Cona”). Table 5 presents a summary of calculated half-lives. Cona MB IgG1 LLRAL showed significantly enhanced PK properties compared to Cona MB IgG1 wt. For the first 7 days, the plasma concentrations of Cona MB IgG1 LLRAL remained comparable to that of conatumumab. The Cona MB IgG1 LLRAL plasma concentration on day 15 was 125× the IC₅₀ value (˜ 0.04 μg/mL) determined in an in vitro cytotoxicity assay (see Example 5) and on day 21, 40× the IC₅₀ value.

FIG. 5C shows plots of plasma concentration over time in a multi-dose PK study for Cona MB IgG1 LLRAL and conatumumab.

TABLE 5
Half-lives in mice after a single dose

| | | Cona MB IgG1 | Cona MB IgG1 wt |
|---|---|---|---|
| Half-life [days] | Cona i.v. | LLRAL i.v. | i.v. |
| 3 h-24 h | 3.5 | 1.0 | 0.9 |
| 24 h-5 days | 5.7 | 5.2 | 1.5 |
| 5 days-15 days | 14.8 | 3.1 | 2.2 |

### Example 7. Therapeutic Effect of DR5-Targeting Multabodies in a Xenograft Mouse Model

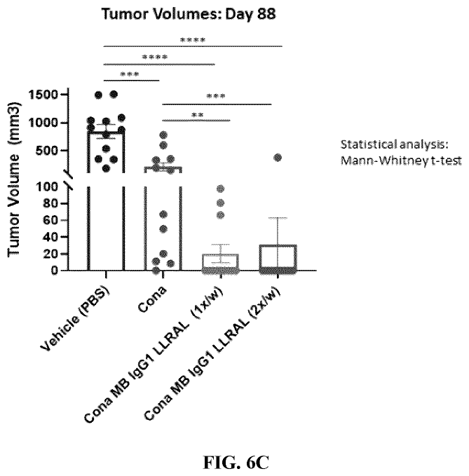

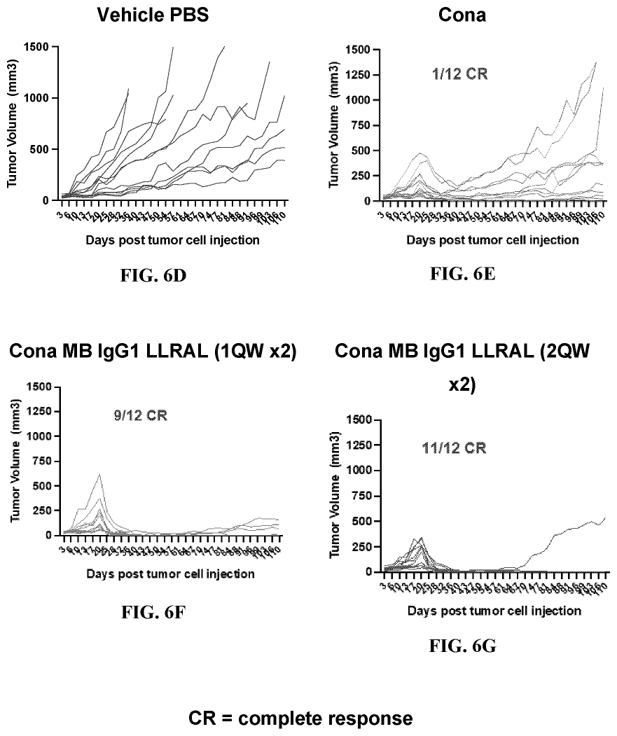

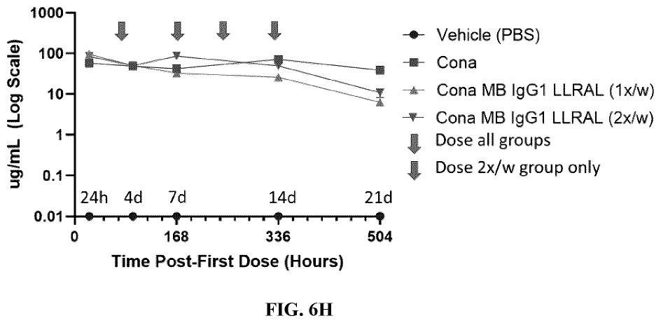

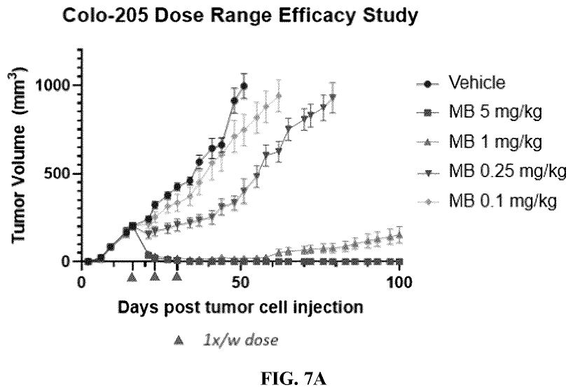

The therapeutic effect of exemplary Multabodies was evaluated in a colon cancer xenograft model. FIG. 6A shows a schematic illustrating the study design of the in vivo efficacy study.

5×10⁶ COLO 205 cells were injected subcutaneously in the flank of BALB/c SCID mice (n=12 per group). Tumors were allowed to grow to an average size of 200 mm³. Mice were sorted into treatment or control groups such that the mean tumor volumes were equal across groups (FIG. 6B). Mice received treatment (5 mg/kg per dose) or control via intraperitoneal (i.p.) injection once or twice weekly for two weeks (FIG. 6A). Tumor volume was measured twice weekly using calipers. Blood samples were collected 24 h, 7 days, 14 days and 21 days following the initial dose, and the drug levels in plasma were measured as described in Example 6.

FIGS. 6B-6G are plots that depict tumor volumes of mice in various groups. The plots in FIGS. 6B and 6D-6G show tumor volumes over time, and the plot in FIG. 6C shows the tumor volume at day 88 after treatment initiation. Treatment with Cona MB IgG1 LLRAL once or twice weekly significantly inhibited the growth of large established tumors, suggesting that Cona MB IgG1 LLRAL was able to penetrate tumors even of large size. Cona MB IgG1 LLRAL inhibited tumor growth more strongly than conatumumab did, resulting in 9/12 and 11/12 complete regressions in the once weekly-treated and twice weekly-treated group, respectfully. This compares to only 1/12 complete remissions in the conatumumab-treated group.

FIG. 6H is a plot showing the concentrations of test molecules in plasma (y-axis) as a function of the time after the first dose (x-axis). The pharmacokinetic profile of Cona MB IgG1 LLRAL as observed in the COLO205 colon cancer mouse model is similar to that observed in the multi-dose PK study described in Example 6. (See FIG. 5B). These results suggest that the presence of tumor does not impact the clearance of the Multabody.

These experiments were repeated in a second study with the same study design, using vehicle, conatumumab, or Cona MB IgG1 LLRAL once weekly for two weeks, at 5 mg/kg per dose. Results from this second study were similar with those from the first study: no tumor regression was observed in the vehicle group, none of the ten mice in the conatumumab group exhibited complete tumor regression, and all ten mice in the Cona MB IgG1 LLRAL group showed complete tumor regression (data not shown). Results from this second study indicate that the therapeutic effect of Cona MB IgG1 LLRAL is highly reproducible with once weekly dosing.

### Example 8. Dose Range Efficacy of DR5-Targeting Multabodies in a Xenograft Mouse Model

The dose range efficacy of Cona IgG1 MB LLRAL was also explored in the same colon cancer xenograft model used in Example 6.

5×10⁶ COLO 205 cells were injected subcutaneously in the flank of Balb/c SCID mice. Tumors were allowed to grow to an average size of 200 mm³. Mice were sorted into one of five groups (vehicle or Cona MB IgG1 LLRAL at 0.1 mg/kg, 0.25 mg/kg, 1 mg/kg, or 5 mg/kg per dose) such that the mean tumor volumes were equal across the groups. Mice received treatment or vehicle via intraperitoneal (i.p.) injection once weekly for three weeks. Tumor volume was measured twice weekly using calipers. Blood samples were obtained throughout the experiment.

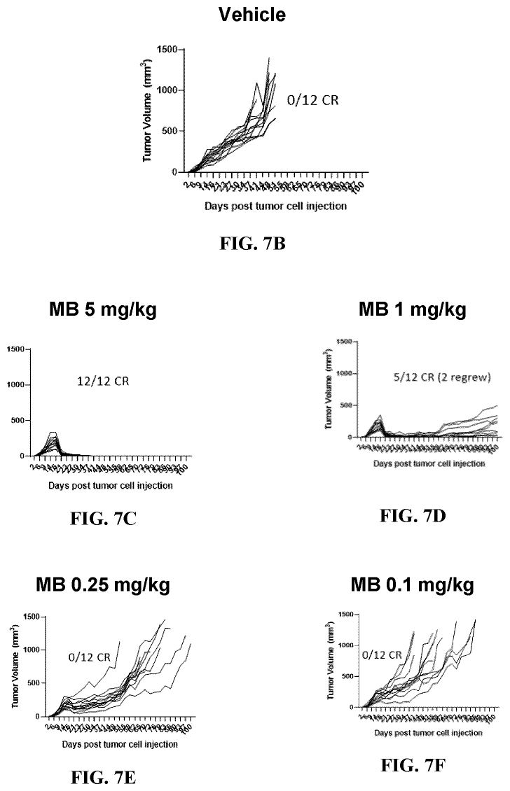

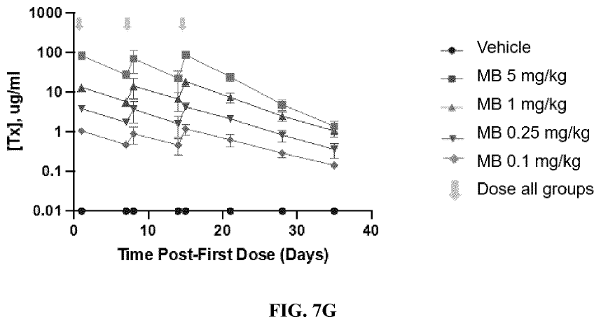

FIG. 7A is a plot that depicts tumor volumes of mice in various groups over time following the first dose. “MB” indicates the Cona MB IgG1 LLRAL group. FIGS. 7B-7F show the tumor volumes of individual mice within the vehicle (FIG. 7B) and 5 mg/kg. 1 mg/kg. 0.25 mg/kg, and 0.1 mg/kg Cona MB IgG1 LLRAL treatment (FIG. 7C, FIG. 7D, FIG. 7E, and FIG. 7F, respectively) groups. As shown in FIG. 7A, mice treated with Cona MB IgG1 LLRAL at 0.25 mg/kg, 1 mg/kg/and 5 mg/kg doses showed significant improvement over the vehicle group. Cona MB IgG1 LLRAL appeared to have similar efficacy at doses of 1 mg/kg and 5 mg/kg initially. However, the 1 mg/kg showed fewer complete responses and more regrowth than did the 5 mg/kg treatment group, in which complete regression was seen in twelve out of twelve mice.

FIG. 7G is a plot that depicts the amount of Cona MB IgG1 LLRAL detectable in blood samples collected at various timepoints after the first dose was administered. Cona MB IgG1 LLRAL was detectable in the plasma at all time points tested in the 0.1 mg/kg. 0.25 mg/kg. 1 mg/kg/and 5 mg/kg Cona MB IgG1 LLRAL treatment groups, with the pharmacokinetics appearing linear in all dose groups.

These results demonstrate that Cona MB IgG1 LLRAL exhibited therapeutic efficacy at multiple doses tested, including comparable efficacy at a dose level that was five-fold lower than that tested in Example 6. However, whereas complete tumor regression was observed at the 5 mg/kg dose level, some tumor regrowth was observed when the dose level was lowered to 1 mg/kg.

### Example 9. Penetration of Large Tumors and Induction of Apoptosis by a DR5-Targeting Multabody

Exemplary Multabodies generated as described in Example 1 were evaluated for their ability to penetrate large tumors and induce apoptosis in a colon xenograft model.

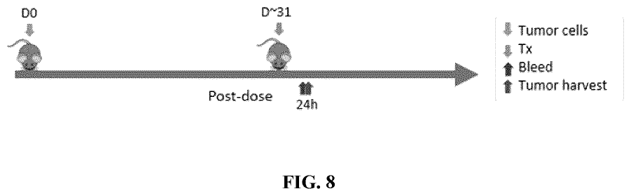

5×10⁶ COLO 205 cells were injected subcutaneously in the flank of Balb/c SCID mice. Tumors were allowed to an average size of 500 mm³. Tumor volume was measured twice weekly using calipers. Mice were randomly assigned to a vehicle or treatment groups such that the average tumor size was the same across the two groups, about 500 mm³ in each group. Mice were then administered one dose of vehicle or 5 mg/kg Cona MB IgG1 LLRAL by intraperitoneal (i.p.) injection. Blood samples were collected 24 hours after injection of vehicle or Cona MB IgG1 LLRAL, and tumors were harvested for subsequent histological analyses. FIG. 8 depicts a schematic and timeline for these experiments.

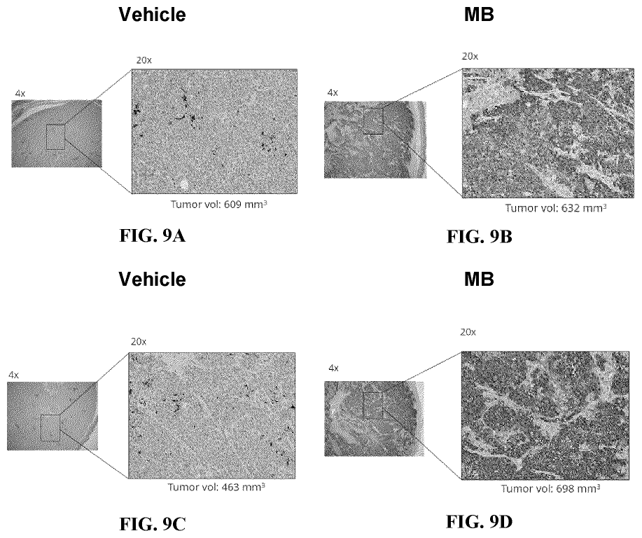

Tumors were fixed in formalin, embedded in paraffin, and sectioned. Tumor sections were stained immunohistochemically for cleaved caspase-3, a marker of apoptosis. Representative images of stained tissue sections are shown in FIGS. 9A and 9C (vehicle-treated mice) and FIGS. 9B and 9D (Cona MB IgG1 LLRAL-treated mice). As shown in these figures, few apoptotic cells were detected in tumor sections from vehicle-treated mice. In contrast, tissue sections from Cona MB IgG1 LLRAL-treated mice contained a large proportion of apoptotic cells throughout the tumor including deep within the core of the tumor, away from the tumor margin (see stained cells in FIGS. 9B and 9D).

These results confirm that Cona MB IgG1 LLRAL induced apoptosis of tumor cells throughout the tumor. Moreover, these results confirm that Multabodies are able to penetrate into the core of large established tumors.

### SEQUENCE LISTING

Underlining within fusion sequences indicate linker sequences.
Bolding within fusion sequences indicate ferritin or ferritin subunit sequences.
Within variable region sequences, underlining and bolding together indicate complementary determining regions sequences.
Boxed and bolded residues indicate residues that are mutated relative to a reference molecule, e.g. relative to an IgG1 Fc.

hFTL
SEQ ID NO: 1
MSSQIRQNYSTDVEAAVNSLVNLYLQASYTYLSLGFYFDRDDVALEGVSHFFRELAEEKREG
YERLLKMQNQRGGRALFQDIKKPAEDEWGKTPDAMKAAMALEKKLNQALLDLHALGSARTDP
HLCDFLETHFLDEEVKLIKKMGDHLTNLHRLGGPEAGLGEYLFERLTLRHD
N_hFTL
SEQ ID NO: 2
MSSQIRQNYSTDVEAAVNSLVNLYLQASYTYLSLGFYFDRDDVALEGVSHFFRELAEEKREG
YERLLKMQNQRGGRALFQDIKKPAEDEW
C_hFTL
SEQ ID NO: 3
GKTPDAMKAAMALEKKLNQALLDLHALGSARTDPHLCDFLETHFLDEEVKLIKKMGDHLTNL
HRLGGPEAGLGEYLFERLTLRHD
IgG1 Fc
SEQ ID NO: 4
DKTHTCPPCPAPELLGGPSVFLFPPKPKDTLMISRTPEVTCVVVDVSHEDPEVKENWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
IgG1 scFc
SEQ ID NO: 5
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGSDKTHTCPPCPAPE
LLGGPSVFLFPPKPKDTLMISRTPEVTCVVVDVSHEDPEVKENWYVDGVEVHNAKTKPREEQ
YNSTYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRWQQ
GNVFSCSVMHEALHNHYTQKSLSLSPGK
Cona LC
SEQ ID NO: 6
EIVLTQSPGTLSLSPGERATLSCRASQGISRSYLAWYQQKPGQAPSLLIYGASSRATGIPDR
FSGSGSGTDFTLTISRLEPEDFAVYYCQQFGSSPWTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSENRGEC
Cona HC
SEQ ID NO: 7
QVQLQESGPGLVKPSQTLSLTCTVSGGSISSGDYFWSWIRQLPGKGLEWIGHIHNSGTTYYN
PSLKSRVTISVDTSKKQFSLRLSSVTAADTAVYYCARDRGGDYYYGMDVWGQGTTVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
Cona scFab
SEQ ID NO: 8
EIVLTQSPGTLSLSPGERATLSCRASQGISRSYLAWYQQKPGQAPSLLIYGASSRATGIPDR
FSGSGSGTDFTLTISRLEPEDFAVYYCQQFGSSPWTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGECGGGGSGGGGSGGGGSGGGGSGGGGSGGGGSGGG
GSGGGGSGGGGSGGGGSGGGGSGGGGSGGGGSGGGGSQVQLQESGPGLVKPSQTLSLTCTVS
GGSISSGDYFWSWIRQLPGKGLEWIGHIHNSGTTYYNPSLKSRVTISVDTSKKQFSLRLSSV
TAADTAVYYCARDRGGDYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKP
SNTKVDKKVEPKSCD
aDR5-hFTL
SEQ ID NO: 9
LEEIVLTQSPGTLSLSPGERATLSCRASQGISRSYLAWYQQKPGQAPSLLIYGASSRATGIP
DRESGSGSGTDFTLTISRLEPEDFAVYYCQQFGSSPWTFGQGTKVEIKRTVAAPSVFIFPPS
DEQLKSGTASVVCLLNNFYPREAKVQWKVDNALQSGNSQESVTEQDSKDSTYSLSSTLTLSK
ADYEKHKVYACEVTHQGLSSPVTKSFNRGECGGGGSGGGGSGGGGSGGGGSGGGGSGGGGSG
GGGSGGGGSGGGGSGGGGSGGGGSGGGGSGGGGSGGGGSQVQLQESGPGLVKPSQTLSLTCT
VSGGSISSGDYFWSWIRQLPGKGLEWIGHIHNSGTTYYNPSLKSRVTISVDTSKKQFSLRLS
SVTAADTAVYYCARDRGGDYYYGMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNH
KPSNTKVDKKVEPKSCDGGGGSGGGGSGGGGSGGGGSGGGGSGGMSSQIRQNYSTDVEAAVN
SLVNLYLQASYTYLSLGFYFDRDDVALEGVSHFFRELAEEKREGYERLLKMQNQRGGRALFQ
DIKKPAEDEWGKTPDAMKAAMALEKKLNQALLDLHALGSARTDPHLCDFLETHFLDEEVKLI
KKMGDHLTNLHRLGGPEAGLGEYLFERLTLRHD
aDR5-hFTL, alternative sequence
SEQ ID NO: 10
EIVLTQSPGTLSLSPGERATLSCRASQGISRSYLAWYQQKPGQAPSLLIYGASSRATGIPDR
FSGSGSGTDFTLTISRLEPEDFAVYYCQQFGSSPWTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGECGGGGSGGGGSGGGGSGGGGSGGGGSGGGGSGGG
GSGGGGSGGGGSGGGGSGGGGSGGGGSGGGGSGGGGSQVQLQESGPGLVKPSQTLSLTCTVS
GGSISSGDYFWSWIRQLPGKGLEWIGHIHNSGTTYYNPSLKSRVTISVDTSKKQFSLRLSSV
TAADTAVYYCARDRGGDYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKP
SNTKVDKKVEPKSCDGGGGSGGGGSGGGGSGGGGSGGGGSGGSSQIRQNYSTDVEAAVNSLV
NLYLQASYTYLSLGFYFDRDDVALEGVSHFFRELAEEKREGYERLLKMQNQRGGRALFQDIK
KPAEDEWGKTPDAMKAAMALEKKLNQALLDLHALGSARTDPHLCDFLETHFLDEEVKLIKKM
GDHLTNLHRLGGPEAGLGEYLFERLTLRHD
aDR5-N_hFTL
SEQ ID NO: 11
EIVLTQSPGTLSLSPGERATLSCRASQGISRSYLAWYQQKPGQAPSLLIYGASSRATGIPDR
FSGSGSGTDFTLTISRLEPEDFAVYYCQQFGSSPWTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGECGGGGSGGGGSGGGGSGGGGSGGGGSGGGGSGGG
GSGGGGSGGGGSGGGGSGGGGSGGGGSGGGGSGGGGSQVQLQESGPGLVKPSQTLSLTCTVS
GGSISSGDYFWSWIRQLPGKGLEWIGHIHNSGTTYYNPSLKSRVTISVDTSKKQFSLRLSSV
TAADTAVYYCARDRGGDYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKP
SNTKVDKKVEPKSCDGGGGSGGGGSGGGGSGGGGSGGGGSGGMSSQIRQNYSTDVEAAVNSL
VNLYLQASYTYLSLGFYFDRDDVALEGVSHFFRELAEEKREGYERLLKMQNQRGGRALFQDI
KKPAEDEW
aDR5-N_hFTL, alternative sequence
SEQ ID NO: 12
EIVLTQSPGTLSLSPGERATLSCRASQGISRSYLAWYQQKPGQAPSLLIYGASSRATGIPDR
FSGSGSGTDFTLTISRLEPEDFAVYYCQQFGSSPWTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGECGGGGSGGGGSGGGGSGGGGSGGGGSGGGGSGGG
GSGGGGSGGGGSGGGGSGGGGSGGGGSGGGGSGGGGSQVQLQESGPGLVKPSQTLSLTCTVS
GGSISSGDYFWSWIRQLPGKGLEWIGHIHNSGTTYYNPSLKSRVTISVDTSKKQFSLRLSSV
TAADTAVYYCARDRGGDYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKP
SNTKVDKKVEPKSCDGGGGSGGGGSGGGGSGGGGSGGGGSGGSSQIRQNYSTDVEAAVNSLV
NLYLQASYTYLSLGFYFDRDDVALEGVSHFFRELAEEKREGYERLLKMQNQRGGRALFQDIK
KPAEDEW
scFc-C_hFTL IgG1 WT
SEQ ID NO: 13
DKTHTCPPCPAPELLGGPSVFLFPPKPKDTLMISRTPEVTCVVVDVSHEDPEVKENWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFEL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGSDKTHTCPPCPAPE
LLGGPSVFLFPPKPKDTLMISRTPEVTCVVVDVSHEDPEVKENWYVDGVEVHNAKTKPREEQ
YNSTYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRWQQ
GNVFSCSVMHEALHNHYTQKSLSLSPGKGGGGSGGGGSGGGGSGGGGSGGGGSGGGKTPDAM
KAAMALEKKLNQALLDLHALGSARTDPHLCDFLETHFLDEEVKLIKKMGDHLTNLHRLGGPE
AGLGEYLFERLTLRHD
scFc-C_hFTL IgG1 LALA
SEQ ID NO: 14

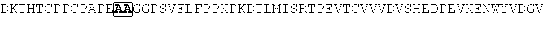

EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGSDKTHTCPPCPAPE

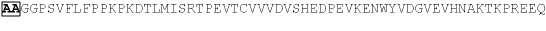

YNSTYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRWQQ
GNVFSCSVMHEALHNHYTQKSLSLSPGKGGGGSGGGGSGGGGSGGGGSGGGGSGGGKTPDAM
KAAMALEKKLNQALLDLHALGSARTDPHLCDFLETHFLDEEVKLIKKMGDHLTNLHRLGGPE
AGLGEYLFERLTLRHD
scFc-C_hFTL IgG1 LALAP
SEQ ID NO: 15

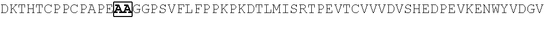

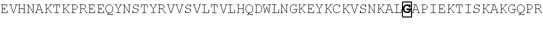

EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGSDKTHTCPPCPAPE

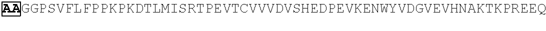

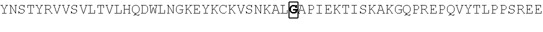

MTKNQVSLTCLVKGFYPSDIAVEWESNGQPENNYKTTPPVLDSDGSFFLYSKLTVDKSRWQQ
GNVFSCSVMHEALHNHYTQKSLSLSPGKGGGGSGGGGSGGGGSGGGGSGGGGSGGGKTPDAM
KAAMALEKKLNQALLDLHALGSARTDPHLCDFLETHFLDEEVKLIKKMGDHLTNLHRLGGPE
AGLGEYLFERLTLRHD
scFc-C_hFTL IgG1 LLRAL
SEQ ID NO: 16

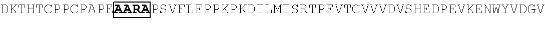

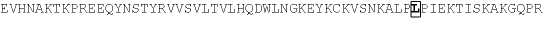

EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGSDKTHTCPPCPAPE

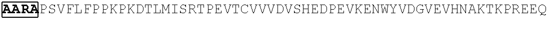

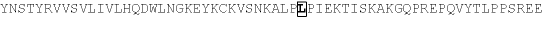

MTKNQVSLTCLVKGFYPSDIAVEWESNGQPENNYKTTPPVLDSDGSFFLYSKLTVDKSRWQQ
GNVFSCSVMHEALHNHYTQKSLSLSPGKGGGGSGGGGSGGGGSGGGGSGGGGSGGGKTPDAM
KAAMALEKKLNQALLDLHALGSARTDPHLCDFLETHFLDEEVKLIKKMGDHLTNLHRLGGPE
AGLGEYLFERLTLRHD
scFc-C_hFTL IgG1 LLGRAL
SEQ ID NO: 17

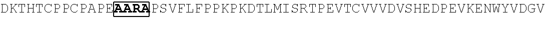

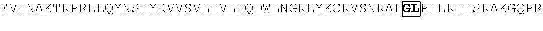

EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGSDKTHTCPPCPAPE

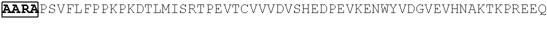

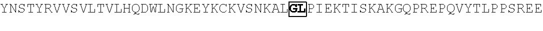

MTKNQVSLTCLVKGFYPSDIAVEWESNGQPENNYKTTPPVLDSDGSFFLYSKLTVDKSRWQQ
GNVFSCSVMHEALHNHYTQKSLSLSPGKGGGGSGGGGSGGGGSGGGGSGGGGSGGGKTPDAM
KAAMALEKKLNQALLDLHALGSARTDPHLCDFLETHFLDEEVKLIKKMGDHLTNLHRLGGPE
AGLGEYLFERLTLRHD
GS linker sequence
SEQ ID NO: 18
GGGS
GS linker sequence
SEQ ID NO: 19
GGGGS

### Equivalents/Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

1. A fusion polypeptide comprising:
(1) an Fc polypeptide and
(2) a nanocage monomer or subunit thereof,
wherein the Fc polypeptide comprises an IgG1 Fc chain, wherein said IgG1 Fc chain comprises (1) an amino acid residue other than glycine at position 237 and (2) a proline residue at position 329, according to EU numbering.

2. The fusion polypeptide of claim 1, wherein the nanocage monomer or subunit thereof is a ferritin monomer or subunit thereof.

3. The fusion polypeptide of claim 2, wherein the ferritin monomer or subunit thereof is a ferritin light chain or subunit thereof.

4. The fusion polypeptide of claim 2 or 3, wherein the ferritin monomer or subunit thereof is a human ferritin or subunit thereof.

5. The fusion polypeptide of any one of claims 2-4, wherein the ferritin monomer or subunit thereof is a ferritin monomer subunit.

6. The fusion polypeptide of claim 5, wherein the ferritin monomer subunit is a C-half ferritin.

7. The fusion polypeptide of claim 6, wherein the Fc polypeptide is linked to the C-half ferritin's N-terminus.

8. The fusion polypeptide of claim 9, wherein the Fc polypeptide is linked to the C-half ferritin's N-terminus via an amino acid linker.

9. The fusion polypeptide of claim 8, wherein the amino acid linker comprises a (GₙS)ₘ linker.

10. The fusion polypeptide of claim 9, wherein the (GₙS)ₘ linker is a (GGGGS)ₘ(SEQ ID NO: 19) linker.

11. The fusion polypeptide of any one of claims 1-10, wherein the Fc polypeptide comprises a single chain Fc (scFc) comprising two Fc chains, wherein the two Fc chains are linked via an amino acid linker.

12. The fusion polypeptide of claim 11, wherein the amino acid linker that links the two Fc chains comprises a (GₙS)ₘ linker.

13. The fusion polypeptide of claim 12, wherein the (GₙS)ₘ linker is a (GGGGS)ₘ(SEQ ID NO: 19) linker.

14. The fusion polypeptide of any one of claims 1-13, wherein the Fc polypeptide comprises an IgG1 Fc chain.

15. The fusion polypeptide of claim 14, wherein the IgG1 Fc chain comprises an alanine at position 237 according to EU numbering.

16. The fusion polypeptide of claim 14 or 15, wherein the IgG1 Fc chain comprises: an alanine at position 234, an alanine at position 235, an arginine at position 236, and a leucine at position 330, according to EU numbering.

17. A self-assembled polypeptide complex comprising:
(a) a plurality of first fusion polypeptides, each first fusion polypeptide being a fusion polypeptide of any one of claims 1-16, and
(b) a plurality of second fusion polypeptides, each second fusion polypeptide comprising (1) an antigen-binding antibody fragment and a (2) a nanocage monomer or subunit thereof.

18. The self-assembled polypeptide complex of claim 17, the nanocage monomer or subunit thereof of each second fusion polypeptide is a ferritin monomer or subunit thereof.

19. The self-assembled polypeptide complex of claim 18, wherein the ferritin monomer or subunit thereof is a ferritin light chain or subunit thereof.

20. The self-assembled polypeptide complex of claim 18 or 19, wherein the ferritin monomer or subunit thereof is a human ferritin or subunit thereof.

21. The self-assembled polypeptide complex of claim 19 or 20, which does not comprise any ferritin heavy chains or subunits of ferritin heavy chains.

22. The self-assembled polypeptide complex of any one of claims 17-21, wherein, within each second fusion polypeptide, the antigen-binding antibody fragment is linked to the nanocage monomer or subunit thereof via an amino acid linker.

23. The self-assembled polypeptide complex of claim 22, wherein the amino acid linker comprises a (GₙS)ₘ linker.

24. The self-assembled polypeptide complex of claim 23, wherein the (GₙS)ₘ linker is a (GGGGS)ₘ(SEQ ID NO:19) linker.

25. The self-assembled polypeptide complex of any one of claims 17-24, wherein the antigen-binding antibody fragment of each second fusion polypeptide is linked to the N-terminus of nanocage monomer or subunit thereof.

26. The self-assembled polypeptide complex of any one of claims 17-25, wherein the antigen-binding antibody fragment of each second fusion polypeptide is a Fab fragment.

27. The self-assembled polypeptide complex of any one of claims 17-26, wherein each second fusion polypeptide does not comprise any antibody CH2 or CH3 domains.

28. The self-assembled polypeptide complex of any one of claims 17-27, further comprising a plurality of third fusion polypeptides, each third fusion polypeptide comprising (1) an antigen-binding antibody fragment and (2) a nanocage monomer or a subunit thereof, wherein the third fusion polypeptide is different than the second fusion polypeptide.

29. The self-assembled polypeptide complex of claim 28, wherein the antigen-binding antibody fragment of each third fusion polypeptide is a Fab fragment.

30. The self-assembled polypeptide complex of claim 29, wherein each third fusion polypeptide does not comprise any antibody CH2 or CH3 domains.

31. The self-assembled polypeptide complex of any one of claims 17-30, wherein the nanocage monomer or subunit thereof of each first fusion polypeptide and each second fusion polypeptide is a ferritin monomer subunit, and
a. each first fusion polypeptide comprises a C-half-ferritin, and each second fusion polypeptide comprises a N-half-ferritin; or
b. each first fusion polypeptide comprises an N-half ferritin, and each second fusion polypeptide comprises a C-half-ferritin.

32. The self-assembled polypeptide complex of any one of claims 17-31, wherein the self-assembled polypeptide complex is characterized by a 1:1 ratio of first fusion polypeptides to second fusion polypeptides.

33. The self-assembled polypeptide complex of any one of claims 17-32, comprising a total of 24 to 48 fusion polypeptides.

34. The self-assembled polypeptide complex of any one of claims 17-32, comprising a total of least 24 fusion polypeptides.

35. The self-assembled polypeptide complex of claim 34, comprising a total of at least 32 fusion polypeptides.

36. The self-assembled polypeptide complex of claim 35, having a total of about 32 fusion polypeptides.

37. The self-assembled polypeptide complex of any one of claims 17-36, which exhibits no binding to at least one human Fcγ receptor, as determined in an in vitro assay.

38. The self-assembled polypeptide complex of claim 37, which exhibits no binding to one or more human Fcγ receptors selected from the group consisting of hFcγRI, hFcγRIIa, hFcγRIIb, hFcγRIIIa, hFcγRIIIb, and combinations thereof, as determined in an in vitro assay.

39. The self-assembled polypeptide complex of claim 38, which exhibits no binding to hFcγRI, as determined in an in vitro assay.

40. The self-assembled polypeptide complex of claim 38 or 39, which exhibits no binding to hFcγRIIa, as determined in an in vitro assay.

41. The self-assembled polypeptide complex of claim 38, 39, or 40, which exhibits no binding to hFcγRIIIa, as determined in an in vitro assay.

42. The self-assembled polypeptide complex of any one of claims 38-41, which exhibits no binding to hFcγRIIb, as determined in an in vitro assay.

43. The self-assembled polypeptide complex of any one of claims 38-42 which exhibits no binding to hFcγRIIIb, as determined in an in vitro assay.

44. A self-assembled polypeptide complex comprising:
(a) a plurality of first fusion polypeptides, each first fusion polypeptide comprising (1) a scFc and (2) a ferritin monomer or subunit thereof, and
(b) a plurality of second fusion polypeptides, each second fusion polypeptide comprising (1) an antigen-binding antibody fragment linked to (2) a ferritin monomer or subunit thereof, wherein the scFc comprises two IgG1 Fc chains, each IgG1 Fc chain comprising: an alanine at position 234, an alanine at position 235, an arginine at position 236, an alanine at position 237, a proline at position 329, and a leucine at position 330, according to EU numbering.

45. The self-assembled polypeptide complex of claim 44, wherein:
a. each first fusion polypeptide comprises a C-half-ferritin, and each second fusion polypeptide comprises an N-half-ferritin; or
b. each first fusion polypeptide comprises an N-half ferritin, and each second fusion polypeptide comprises a C-half-ferritin.

46. The self-assembled polypeptide complex of claim 45, wherein each first fusion polypeptide comprises a scFc linked to the C-half-ferritin's N-terminus, and each second fusion polypeptide comprises a Fab linked to the N-half-ferritin's N-terminus.

47. The self-assembled polypeptide complex of claim 46, wherein, (1) in each first fusion polypeptide, the scFc is linked via an amino acid linker to the C-half ferritin's N-terminus and/or (2) in each second fusion polypeptide, the Fab is linked via an amino acid linker to N-half ferritin's N-terminus.

48. The self-assembled polypeptide complex of any one of claims 45-47, wherein the self-assembled polypeptide complex is characterized by a 1:1 ratio of first fusion polypeptides to second fusion polypeptides.

49. The self-assembled polypeptide complex of any one of claims 44-48, further comprising a plurality of third fusion polypeptides, each third fusion polypeptide comprising (1) an antigen-binding antibody fragment linked to (2) a nanocage monomer or a subunit thereof, wherein the third fusion polypeptide is different than the second fusion polypeptide.

50. The self-assembled polypeptide complex of any one of claims 44-49, comprising a total of 24 to 48 fusion polypeptides.

51. The self-assembled polypeptide complex of any one of claims 44-50, comprising a total of least 24 fusion polypeptides.

52. The self-assembled polypeptide complex of claim 51, comprising a total of at least 32 fusion polypeptides.

53. The self-assembled polypeptide complex of claim 52, having a total of about 32 fusion polypeptides.

54. The self-assembled polypeptide complex of any one of claims 17-53, wherein the antibody-binding fragment is capable of binding to a tumor-associated antigen or an antigen associated with an autoimmune disorder.

55. A method comprising administering a composition comprising the self-assembled polypeptide complex of any one of claims 17-54 to a mammalian subject.

56. The method of claim 55, wherein the subject is human.

57. The method of claim 55 or 56, wherein the subject has or is at risk of developing cancer.

58. The method of claim 55 or 56, wherein the subject has or is at risk of developing an autoimmune disorder.

59. The method of any one of claims 55-58, comprising administration by a systemic route.

60. The method of claim 59, wherein the systemic route comprises subcutaneous, intravenous, or intramuscular injection, inhalation, or intranasal administration.

61. Use of a composition comprising the self-assembled polypeptide complex of any one of claims 17-54 for administration to a mammalian subject.

62. The use of claim 61, wherein the subject is human.

63. The use of claim 61 or 62, wherein the subject has or is at risk of developing cancer.

64. The use of claim 61 or 62, wherein the subject has or is at risk of developing an autoimmune disorder.

65. The use of any one of claims 61-64, for administration by a systemic route.

66. The use of claim 65, wherein the systemic route comprises subcutaneous, intravenous, or intramuscular injection, inhalation, or intranasal administration.

67. A composition comprising the self-assembled polypeptide complex of any one of claims 17-54 for use in administration to a mammalian subject.

68. The composition for use of claim 67, wherein the subject is human.

69. The composition for use of claim 67 or 68, wherein the subject has or is at risk of developing cancer.

70. The composition for use of claim 67 or 68, wherein the subject has or is at risk of developing an autoimmune disorder.

71. The composition for use of any one of claims 67-70, for administration by a systemic route.

72. The composition for use of claim 71, wherein the systemic route comprises subcutaneous, intravenous, or intramuscular injection, inhalation, or intranasal administration.


SEQUENCE LISTING (19 sequences)

SEQ ID NO: 1 - protein, 175 aa, synthetic construct
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 2 - protein, 90 aa, synthetic construct
MSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 3 - protein, 85 aa, synthetic construct
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 4 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 524 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 215 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 452 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 511 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 9 - protein, 715 aa, synthetic construct
LEEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 10 - protein, 712 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 11 - protein, 628 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 12 - protein, 627 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 13 - protein, 636 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 14 - protein, 636 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 15 - protein, 636 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 16 - protein, 636 aa, synthetic construct
DKTHTCPPCPAPEAARAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAARAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 17 - protein, 636 aa, synthetic construct
DKTHTCPPCPAPEAARAPSVFLFPPKPKDTLMISRTPEVTCVVVDVSHEDPEVKFNWYVDGVEVHNAKTKPREEQYNSTYRVVSVLTVLHQDWLNGKEYKCKVSNKALGLPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAARAPSVFLFPPKPKDTLMISRTPEVTCVVVDVSHEDPEVKFNWYVDGVEVHNAKTKPREEQYNSTYRVVSVLTVLHQDWLNGKEYKCKVSNKALGLPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 18 - protein, 4 aa, synthetic construct
GGGS

SEQ ID NO: 19 - protein, 5 aa, synthetic construct
GGGGS